SEQUENCE LISTING (11 sequences)

SEQ ID NO: 1 - protein, 8 aa, X. laevis
SSSQVSPA

SEQ ID NO: 2 - protein, 25 aa, X. laevis
DEDGSSAATSKTEASSVSSSQVSPA

SEQ ID NO: 3 - protein, 44 aa, X. laevis
KQFRNCLITTLCCGKNPFGDEDGSSAATSKTEASSVSSSQVSPA

SEQ ID NO: 4 - protein, 8 aa, Homo sapiens
ETSQVAPA

SEQ ID NO: 5 - protein, 9 aa, Homo sapiens
TETSQVAPA

SEQ ID NO: 6 - protein, 15 aa, Bos taurus
STTVSKTETSQVAPA

SEQ ID NO: 7 - DNA, 34 bp, Bacterio-phage P1
ataacttcgtatagcatacattatacgaagttat

SEQ ID NO: 8 - DNA, 29 bp, Artificial Sequence
  synthetic primer
gccgccaccatggaggaatgctgggtgac

SEQ ID NO: 9 - DNA, 49 bp, Artificial Sequence
  synthetic primer
ttaggctggagccacctggctggtctccgtcttggaagcggtggcagag

SEQ ID NO: 10 - protein, 15 aa, Mus musculus
SATASKTETSQVAPA

SEQ ID NO: 11 - protein, 18 aa, Xenopus laevis
ATSKTEASSVSSSQVSPA

(19) United States
(12) Patent Application Publication
(10) Pub. No.: US 2005/0091702 A1
(43) Pub. Date: Apr. 28, 2005

(54) Expression of polypeptides in rod outer segment membranes

(76) Inventors: Krzysztof Palczewski, Bellevue, WA (US); Ning Li, San Diego, CA (US); Juan Ballesteros, Encinitas, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/831,773

(22) Filed: Apr. 23, 2004

Related U.S. Application Data
(63) Continuation-in-part of application No. 09/990,185, filed on Nov. 21, 2001.

(51) Int. Cl. A01K 67/00; C12N 15/85

(52) U.S. Cl. 800/8; 435/320.1; 435/455; 435/325

(57) ABSTRACT

The invention provides a transgene construct consisting of a transgene encoding a membrane polypeptide comprising a rod outer segment (ROS) targeting signal, and an operably associated species specific rod-specific regulatory sequence, wherein said polypeptide is not rhodopsin. The membrane polypeptide can be a G protein-coupled receptor (GPCR). Transgenic membrane polypeptides include a receptor selected from 5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆, 5HT_{7A}, EDG1, EDG2, EDG3, EDG4, EDG5, EDG6, EDG7, EDG8, CB2, FMLP and MC4. The invention also provides a Xenopus cell whose genome contains a transgene encoding a membrane polypeptide comprising a ROS targeting signal operably associated with a Xenopus rod-specific regulatory sequence, wherein said polypeptide is not a rhodopsin. A Xenopus tadpole or adult whose genome contains a transgene encoding a membrane polypeptide comprising a ROS targeting signal operably associated with a Xenopus rod-specific regulatory sequence, wherein said polypeptide is not a rhodopsin is further provided.

This application is a continuation-in-part application of U.S. Ser. No. 09/990,185, filed Nov. 21, 2001, and which is incorporated herein by reference in its entirety.

This invention was made with government support under grant number 1 R44 MH68919-01 and N43-CM-37011 awarded by the National Institutes of Health. The United States Government has certain rights in this invention.

### BACKGROUND OF THE INVENTION

The invention relates generally to the fields of protein structural biology and pharmaceutical design and, more specifically, to DNA constructs, cells and animals suitable for producing and isolating homogeneous proteins.

Membrane proteins are critical for cellular communication, electrical and ion balance, structural integrity of cells, cell adhesion, and other functions. Among membrane proteins, G-protein coupled receptors (GPCRs) are of particular interest, because they form one of the largest and most diverse groups of receptor proteins. The more than 400 nonosensory GPCRs in the human genome are involved in the regulation of a multitude of physiological process. Several hundred other GPCRs are involved in sensing light, odor and taste. More than 40% of the total sales of available drugs are aimed at GPCRs, and GPCRs are being actively investigated throughout the pharmaceutical industry.

Structural models of proteins have proven useful in predicting mechanisms of ligand binding, predicting the effect of disease-causing mutations, and supporting drug design. However, obtaining atomic resolution structures of membrane proteins has proven technically challenging, in large part because expression of membrane proteins in tissue culture systems, which has conventionally been used to obtain the desired protein in large amounts, yields proteins that lack certain of the post-translational modifications found in native proteins, such as fatty acylation, phosphorylation and N— and O-linked glycosylation, or that have altered patterns of such modifications compared to native proteins. These differences can affect the stability of the protein, making it hard to isolate in soluble form. Additionally, the exact post-translational modifications differ from molecule to molecule in tissue culture systems. This heterogeneity detrimentally affects the ability to form suitable crystals for structural studies. For example, the GPCR bovine rhodopsin, purified either from recombinant mammalian cell lines or baculovirus/insect cells, exhibits differences in the amount of N-glycosylation as compared to rhodopsin isolated from bovine rod cells, and also exhibits a more diffuse band on an electrophoresis gel, indicative of heterogeneity (Reeves et al., Proc. Natl. Acad Sci. USA 93:11487-11492 (1996)).

To date, only a single GPCR crystal structure has been determined, that of bovine rhodopsin (Palczewski et al., Science 289:739-745 (2000)). Rhodopsin is a GPCR involved in the transmission of light signals in the retina. To prepare high quality crystals, rhodopsin was isolated from the membranes of the rod outer segment of bovine retinas, where it constitutes about 90% of the total protein content.

Unfortunately, natural sources of most other membrane proteins in similar abundance and purity are not available. Additionally, as described above, proteins isolated from recombinant sources in tissue culture are generally heterogeneous and thus have not proven suitable for structural studies. Thus, there exists a need for a method for producing proteins, and particularly membrane proteins such as GPCRs, in high abundance, purity and homogeneity. Such proteins can be used for structural studies as well as for other research and therapeutic applications. The present invention satisfies this need and provides related advantages as well.

### SUMMARY OF THE INVENTION

The invention provides a transgene construct consisting of a transgene encoding a membrane polypeptide comprising a rod outer segment (ROS) targeting signal, and an operably associated species specific rod-specific regulatory sequence, wherein said polypeptide is not rhodopsin. The membrane polypeptide can be a G protein-coupled receptor (GPCR). Transgenic membrane polypeptides include a receptor selected from 5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆, 5HT_{7A}, EDG1, EDG2, EDG3, EDG4, EDG5, EDG6, EDG7, EDG8, CB2, FMLP and MC4. The invention also provides a Xenopus cell whose genome contains a transgene encoding a membrane polypeptide comprising a ROS targeting signal operably associated with a Xenopus rod-specific regulatory sequence, wherein said polypeptide is not a rhodopsin. A Xenopus tadpole or adult whose genome contains a transgene encoding a membrane polypeptide comprising a ROS targeting signal operably associated with a Xenopus rod-specific regulatory sequence, wherein said polypeptide is not a rhodopsin is further provided.

### BRIEF DESCRIPTION OF THE DRAWINGS

FIG. 1 shows an exemplary gene targeting construct and targeting strategy for expressing a transgenic polypeptide in the rod outer segment (ROS) membrane of mouse retina. The genomic clone contains five exons (E1 to E5) of the mouse rhodopsin gene (also known as the opsin gene or rod opsin gene). The transgene is a G-protein coupled receptor (GPCR) tagged on its C-terminus with a ROS targeting signal (hatched box). Expression of the transgene is under the control of the mouse rhodopsin promoter (5′ of the arrow). An excisable positive selection marker (neo flanked by loxP sites) and a negative selection marker (DTÿ) are indicated.

FIG. 2 shows a schematic diagram of an exemplary construct for expressing the human cannabinoid receptor 2 (CB2) in the rod outer segment of transgenic Xenopus laevis.

FIG. 3 shows a transgenic construct and use of rod cells as a bioreactor for heterologous polypeptide production.

FIG. 4 shows a Xenopus laevis transgenic construct for heterologous polypeptide expression.

FIG. 5 shows the expression of 5HT_{1A}-GFP-1D4 fusion protein in transgenic Xenopus laevis tadpoles by fluorescence.

FIG. 6 shows the efficiency of transgenic expression of 12 5HT GPCRs in the retina of transgenic Xenopus laevis tadpoles.

FIG. 7 shows the expression of 5HT_{1A}-GFP-1D4 fusion protein in rod cells of transgenic Xenopus laevis tadpoles by microscopy of an eye cryosection.

FIG. 8 shows the expression of 12 different 5HTR-GFP-1D4 fusion protein in rod cells of transgenic Xenopus laevis tadpoles by microscopy of an eye cryosection.

FIG. 9 shows protein blots of extracts from HEK293 cells expressing 12 human 5HT receptors and silver staining of purified receptors.

FIG. 10 shows fractions collected during purification of 5-HT₆ from transfected HEK293 cells.

FIG. 11 shows several protein blots of Xenopus eyes extract expressing 5-HT_{1A}R-GFP-1D4 fusion protein, using different primary antibodies.

FIG. 12 shows a silver stained gel of total membrane protein of transgenic tadpole eyes (left lane) and purified 5HT1A-GFP-1D4 (right lane).

FIG. 13 shows quantification of the expression of 5HT_{1A}R-GFP-1D4 fusion protein in transgenic tadpoles.

FIG. 14 shows total (top) and specific binding (bottom) of a 5HT1A selective ligand to transgenic tadpole membranes expressing the 5HT_{1A} receptor and control tadpoles.

FIG. 15 shows [³⁵S]GTPγS binding to membranes expressing 5HT_{1A}R and controls derived from CHO cells (top) and transgenic tadpoles eyes (bottom).

FIG. 16 shows an immunoaffinity purification at high concentration of functional rhodopsin from an ROS extract.

### DETAILED DESCRIPTION OF THE INVENTION

The invention provides animals that express transgenic polypeptides in the outer segment membrane of rod cells, as well as cells and constructs suitable for preparing such animals. The invention constructs can advantageously be designed so that homozygous animals produce little or no endogeous rhodopsin in the rod cells. The trangenic polypeptides expressed in the rod outer segment (ROS) membranes thus comprise a large percentage of the total ROS membrane protein content, and can be readily purified in large amounts. The transgenic polypeptides are also substantially homogenous in their post-translational modifications. Therefore, polypeptides produced by the invention animals and methods are useful for structural studies to elucidate their molecular mechanisms and ligand interactions, thereby providing useful information for drug design. The ROS membrane-expressed proteins are also useful in other applications known in the art for which high quality protein preparations are required or advantageous, including functional studies; screening for ligands, agonists and antagonists; preparation of antibodies; and preparation of pharmaceuticals.

In one embodiment, the invention provides a gene targeting construct that contains a transgene encoding a polypeptide comprising a rod outer segment (ROS) targeting signal. The transgene is flanked by 5′ and 3′ DNA sequences which are homologous to a rhodopsin (also known as opsin or rod opsin) gene. Following homologous recombination between the construct and a rhodopsin allele, the transgene and a rod-specific regulatory sequence are operably associated and the rhodopsin allele is functionally disrupted. An invention gene targeting construct can advantageously be used, for example, to prepare animals that express the polypeptide encoded by the transgene in the rod outer segment membrane, and to prepare suitable ES cells for use in making such animals.

As used herein, the term “transgene” refers to a DNA sequence which does not naturally occur at the rhodopsin gene locus. A transgene can encode any polypeptide for which expression in the rod outer segment membrane is desirable and for which an encoding sequence is known or can be determined. A large number of nucleotide sequences that encode human and non-human polypeptides are known in the art (see, for example, GenBank and other sequence databases), and others can be readily determined. Suitable coding portions, together with untranslated sequences important for mRNA stability and translation, can be sythesized or cloned by standard recombinant molecular biology methods (see, for example, Sambrook et al., Molecular Cloning: A Laboratory Manual, 3rd ed., Cold Spring Harbor Press, Plainview, N.Y. (2001); Ausubel et al. Current Protocols in Molecular Biology, John Wiley & Sons, New York (most recent supplement); and the like).

As an example, the transgene can encode a G-protein coupled receptor (GPCR), such as a GPCR from a human, a non-human mammal, a non-mammalian vertebrate, an invertebrate (e.g. an insect or nematode), a yeast, a bacteria or a plant. GPCRs are seven-transmembrane-domain polypeptides that transduce G-protein coupled signals in response to ligands. The natural ligands of different GPCRs include peptides, biogenic amines, glycoproteins, nucleotides, ions, lipids, amino acids, light and odorants. Structurally, GPCRs can be divided into three major subfamilies, each of which currently includes orphan receptors as well as receptors whose ligands are characterized (reviewed in Gether, Endocrine Reviews 21:90-113 (2000)).

Exemplary members of the Rhodopsin/ÿ2 adrenergic receptor-like family of GPCRs include receptors for biogenic amines (adrenergic, serotonin, dopamine, muscarinic, histamine and the like), CCK, endothein, tachykinin, neuropeptide Y, TRH, neurotensin, bombesin, growth hormone secretagogues, vertebrate and invertebrate opsins, bradykinin, adenosine, cannabinoid, melanocortin, olfactory signals, chemokines, fMLP, c5A, GnRH, eicosanoid, leukotriene, FSH, LH, TSH, fMLP, galanin, nucleotides, opioids, oxytocin, vasopressin, somatostatin and melatonin, as well as GPCRs activated by proteases.

Exemplary members of the Glucagon/VIP/Calcitonin receptor-like family of GPCRs include receptors for calcitonin, CGRP, CRF, PTH, PTHrP, glucagon, glucagon-like peptide, GIP, GHRH, PACAP, VIP, secretin and latrotoxin.

Exemplary members of the Metabotropic neurotransmitter/Calcium receptor family of GPCRs include metabotropic glutamate receptors, metabotropic GABA receptors, calcium receptors, vomeronasal pheromone receptors and taste receptors.

A database containing links to the nucleotide and amino acid sequences of numerous mammalian GPCRs, including orphan GPCRs, is available at http://www.darmstadt.gmd.de/˜gpcrdb/ or at http://www.gpcr.org. The invention can be practiced with a transgene encoding any GPCR, including variants and mutants of known GPCRs, or any desired fragment thereof.

For example, more than 300 non-sensory GPCRs in the human genome are involved in the regulation of a multitude of physiological process. Of particular interest is the family of serotonin (5-hydroxytryptamine, 5-HT) GPCRs (5HTRs) because 5-HT is a neurotransmitter that has been implicated in the aetiology of numerous disease states, including depression, anxiety, social phobia, schizophrenia, obsessive-compulsive and panic disorders, migraine, hypertension, obesity, eating disorders, vomiting, and irritable bowel syndrome. Therefore, the serotonergic system, and these receptors in particular, are primary targets for intervention therapeutic agents to treat a wide variety of disorders, including mental disorders. Despite their biomedical interest, the only GPCR crystal structure available is that of rhodopsin, the photoreceptor expressed in retina rod cells responsible for light sensitivity. The compositions and methods of the invention can be used to produce substantial quantities of any of these GPCRs as well as other heterologous polypeptides of interest for the identification, development and/or improvement of therapeutic agents.

In this regard, broad range of brain functions are influenced by 5HT, including sleep, cognition, sensory perception, motor activity, temperature regulation, nociception, appetite, sexual behavior, and hormone secretion (see Goodman, L. and A. Gilman, The pharmacological basis of therapeutics, 10th edition). Accordingly, 5-HT receptors (5HTRs) are primary targets for the discovery of psychiatric drugs, including antipsychotic and anxiety drugs. Production of 5HT GPCRs using the compositions and methods of the invention can be used for the development or improvement of therapeutic agents for schizophrenia, depression, and anxiety, for example.

With at least thirteen distinct members grouped into 7 subfamilies, 5-HT receptors (5HTRs) represent one of the most complex families of neurotransmitter receptors. The 5-HT₁ receptor class consists of the five receptors 5-HT_{1A}, 5-HT_{1B}, 5-HT_{1D}, 5-HT_{1E}, and 5-HT_{1F} that share 40-63% overall sequence identity in humans and couple preferentially to G_{i/o} to inhibit cAMP formation. The 5-HT₂ receptors consist of 5-HT_{2A}, 5-HT_{2B} and 5-HT_{2C} and exhibit 46-50% overall sequence identity and couple preferentially to G_{q/11} to increase inositol phosphates and cytosolic [Ca²⁺]. The 5-HT₃ receptors are ligand-gated ion channels. Two subtypes of the 5-HT₅ receptor class have been identified, 5-HT_{5A} and 5-HT_{5B}, and share 70% overall sequence identity in rodents. The human 5-HT_{5B} receptor gene presumably does not encode a functional protein due to the presence of stop codons in its coding sequence (see Matthes, H., et al., Mol Pharmacol, 43:313-9 (1993) and Grailhe, R., et al. Eur J Pharmacol, 418:157-67 (2001)). Human recombinant 5-HT_{5A} receptors show negative coupling to cAMP via Gᵢ and Gₒ (see Francken, B. J., et al., Eur J Pharmacol, 361:299-309 (1998) and Francken, B. J., et al., Mol Pharmacol, 57:1034-44 (2000)), although the receptor also can couple positively to cAMP. Receptors 5-HT₄, 5-HT₆ and 5-HT₇ couple preferentially to Gₛ and promote cAMP formation and share overall sequence identities of less than about 35 percent.

Transgenic expression, purification and demonstration of functional binding characteristics of exemplary 5-HT GPCRs, including 5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆ and 5HT_{7A} has been performed. Other exemplary receptors include CB2 (cannabinoid-2) receptor, FMLP (N-formyl-methionyl-leucyl-phenylalanine peptide or N-formyl peptide) receptor, and MC4 (melanocortin-4) receptor.

Sphingosine-1-phosphate (S1P) and lysophosphatidic acid (LPA) are blood-borne lysophospholipids with a wide spectrum of biological activities, which include stimulation of cell growth, prevention of apoptosis, regulation of actin cytoskeleton, and modulation of cell shape, cell migration, and invasion. Activated platelets appear to be a major source of both S1P and LPA in blood. Despite the diversity of their biosynthetic origins, they are considered to share substantial structural similarity. Indeed, recent investigation has revealed that S1P and LPA act via a single family of G protein-coupled receptors designated as Endothelial differentiation gene receptors (EDG). Each receptor isoform displays a unique tissue expression pattern and coupling to a distinct set of heterotrimeric G proteins, leading to the activation of an isoform-specific panel of multiple intracellular signaling pathways. Recent studies on knock-out mice have unveiled non-redundant EDG receptor functions that are essential for normal development and vascular maturation. CDNAs of EDG1(Genbank accession number AF022137), EDG2(U78192), EDG3(AF022139), EDG4(AF233092), EDG5 (AF034780), EDG6 (AJ000479), EDG7 (AF1 86380) and EDG8 (AF317676) have been cloned. Other exemplary receptors include CB2 (cannabinoid-2, X74328) receptor, FMLP (N-formyl-methionyl-leucyl-phenylalanine peptide or N-formyl peptide, M37128) receptor, and MC4 (melanocortin-4, L08603) receptor.

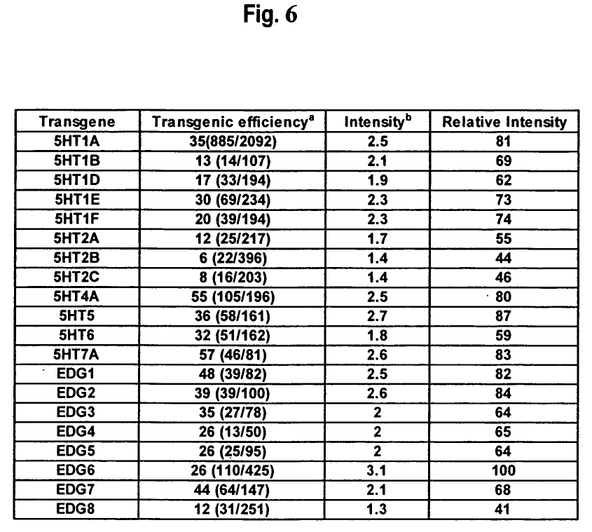

The results obtained for the 5-HT receptors in Xenopus are described further below in the Examples. These results are exemplary of transgenic expression using the compositions and methods of the invention. For example, the CB2 (cannabinoid-2) receptor, FMLP (N-formyl-methionyl-leucyl-phenylalanine peptide or N-formyl peptide) receptor, MC4 (melanocortin-4) receptor and all of the EDG receptors also have been expressed using non-targeted transgenic GFP fusion construct expression and yielded similar results. Transgenic expression results for the 5-HT and EDG receptors are shown in FIG. 6.

Alternatively, the invention can be practiced with a transgene that encodes a membrane protein other than a GPCR. Membrane proteins include receptors for cytokines, growth factors and hormones, including platelet-derived growth factor, epidermal growth factor, insulin, insulin-like growth factor, hepatocyte growth factor, fibroblast growth factor, interleukins, interferons and the like. Membrane proteins also include adhesion molecules, such as an integrins, cadherins and the like; immune molecules, such as antibodies and antigen-binding fragments thereof, T-cell receptors, MHC molecules, cell surface determinants and the like; ion channels; transporters; membrane proteases; death receptors; nuclear receptors; multi-drug resistant proteins; membrane cyclases; tyrosine kinases; membrane phosphatases; or gap junction proteins.

The invention can also be practiced with a transgene that encodes a polypeptide that is not normally membrane localized. For such applications, a membrane localization signal will generally be included within the transgenic polypeptide or within the ROS targeting signal, as described further below. Therefore, the transgene can encode any polypeptide of interest, such as an enzyme (e.g. a kinase, phosphatase, nuclease, protease, polymerase, and the like); binding protein (e.g. a transcription factor, docking protein, receptor agonist or antagonist, and the like); or structural protein (e.g. a cytoskeletal protein, scaffold protein and the like).

Polypeptides expressed in the ROS membrane advantageously have relatively homogeneous post-translational modifications. Accordingly, the invention can be practiced with transgenes that encode polypeptides with extensive post-translation modifications, including multiple disulfide bonds, N— or O-linked glycosylation, fatty acylation, or phosphorylation.

A suitable transgene can encode a naturally occurring polypeptide, including the exemplary polypeptides listed above, from any species of interest, such as human, non-human mammal, other vertebrate, insect, nematode, other invertebrate, plant, yeast, other eukaryote, bacteria or other prokaryote. Advantageously, the transgene can encode a polypeptide having mutations associated with human genetic diseases, such that the structural or functional consequences of these mutations can be determined.

The transgene can also encode a non-naturally occurring polypeptide, such as a polypeptide that contains one or more amino acid additions, deletions or substitutions relative to a naturally occurring sequence. Such variant polypeptides can be used, for example, to characterize the critical functional residues of the polypeptides, such as ligand and effector binding sites, and to aid in the design of suitable therapeutic ligands. Alternatively, a non-naturally occurring polypeptide can consist only of a particular fragment or domain of interest to facilitate structural or functional studies of the particular region.

A transgenic polypeptide can also optionally include additional sequences at internal, N-terminal or C-terminal positions that confer advantageous properties. Such sequences can include, for example, sequences that confer membrane localization on the polypeptide; that facilitate isolation or identification of the polypeptide; or that modulate the function, stabilize the structure or facilitate the folding of the polypeptide.

For example, as described below, a rod outer segment (ROS) targeting signal functions in conjunction with a membrane localization signal to localize a polypeptide to the ROS membrane. Accordingly, if the polypeptide does not normally contain a membrane localization signal, the transgene can be modified by recombinant methods such that the encoded polypeptide will include a membrane localization signal. Suitable membrane localization signals and methods for their use in preparing recombinant polypeptides are well known in the art, and include, for example, myristoylation signals, palmitoylation signals, farnesylation signals, prenylation signals, GPI anchor signals and transmembrane spanning sequences.

Suitable sequences that facilitate identification or isolation of a transgenic polypeptide are known in the art, and can include epitope tags (e.g. HA, myc, FLAG) for which antibodies are available or can be produced, glutathione-S-transferase, poly-His sequences, fluorescent tags (e.g. green fluorescent protein), bioluminescent tags (e.g. luciferase), and the like. Exemplary vector constructions and configurations employing green fluorescent protein (GFP) for facilitating visualization and localization of the expressed transgene are described further below in the Examples. Various other tags and fusion protein configurations can similarly be employed for use in the compositions or methods of the invention. Such other configurations are well known to those skilled in the art.

Sequences that modulate the function, stabilize the structure or facilitate the folding of a transgenic polypeptide include, for example, sequences corresponding to molecules that normally function as ligands, adaptors, effectors or scaffold molecules. By expressing these sequences as fusions with the polypeptide of interest, close proximity of the two molecules and an appropriate stoichiometry are ensured. Additionally, the fused sequences can stabilize the polypeptide in its active or inactive configuration, as desired for a particular application, allowing identification of structural features important for activation.

For example, a GPCR can be expressed as a fusion with its peptide ligand, with an arrestin, or with a G-protein α-subunit. Methods of recombinantly preparing functional GPCR-Gα fusions are known in the art (reviewed in Seifert et al., Trends Pharmacol. Sci. 20:383-389 (1999)). Constructs encoding other desired fusion proteins can be made by routine molecular biological methods.

For certain applications, it may be advantageous to construct a transgene that encodes two or more polypeptides that contain ROS targeting signals, either as separate translation products or as fusions. For example, a transgene can encode two or more different receptor polypeptides that contain ROS targeting signals, such as two or more different GPCRs. Likewise, a transgene can encode one polypeptide that contains a ROS targeting signal and another polypeptide that contains a ROS targeting signal, wherein the two polypeptides are normally associated. Thus, one polypeptide can be a ligand, adaptor, effector or scaffold molecule of the other polypeptide, as described above. For example, one polypeptide can be an arrestin or Gα subunit, while the other polypeptide is a GPCR. The two or more polypeptides can advantageously be used together, such as in screening assays described herein, or isolated from each other by methods known in the art, such as by proteolytic cleavage between fused sequences, or by immunological separation methods.

In certain embodiments, the polypeptide encoded by the transgene in the targeting constructs, cells or animals will not be a rhodopsin. As used with respect to excluded polypeptides, the term “rhodopsin” refers to a naturally occurring rhodopsin polypeptide from any species, as well as any variant or mutant forms thereof described in the art as of the priority date of the application. The term “rhodopsin” is used herein to refer either to the ap oprotein, which is also known as rod opsin, and the protein with the covalently attached chromophore. An excluded rhodopsin contains the rhodopsin N-terminal amino acid sequences and the C-terminal ROS targeting signal as contiguous sequences. Unless specifically indicated, an excluded rhodopsin polypeptide is not a rhodopsin fused to a heterologous polypeptide, such as arrestin or a Gÿ subunit.

Examples of specifically excluded rhodopsins include wild-type Xenopus, mouse, rat, human, pig and bovine rhodopsins, as well as mutant rhodopsins that serve as animal models of retinal disorders such as retinitis pigmentosa (P23H, V20G, P27L; various C-terminal deletions and substitutions), photoreceptor degeneration (K296E), and congenital nightblindness (G90D) (Frederick et al., Invest. Opthalamol. Vis. Sci. 42:826-833 (2001); Li et al., Proc. Natl. Acad. Sci. USA 92:3551-3555 (1995); Sieving et al., J. Neurosci. 21:5449-5460 (2001); and the like).

In an invention targeting construct, the transgene is flanked by 5′ and 3′ DNA sequences that are homologous to the rhodopsin gene from the animal species of interest. Conveniently, the animal species is a mouse. However, it is contemplated that the invention can be practiced with rhodopsin genes from other species amenable to gene targeting procedures, such as rat, guinea pig, bovine, Xenopus, Zebrafish, human, pig, sheep, goat, cat, dog and non-human primate.

A flanking nucleotide sequence that is “homologous” to a rhodopsin gene sequence refers to a nucleotide sequence having sufficient identity to a rhodopsin gene sequence to allow for homologous recombination between the nucleotide sequence and an endogenous rhodopsin gene sequence in a host cell. Typically, the nucleotide sequences of the flanking homology regions are at least 90%, such as at least 95%, 98%, 99% or 100% identical to the nucleotide sequences of the endogenous rhodopsin gene to be targeted for homologous recombination. Advantageously, to enhance the homologous recombination frequency the flanking homologous regions can be isogenic with the targeted endogenous allele, which means that the DNA of the flanking regions is isolated from cells of the same genetic background as the cell into which the targeting construct is to be introduced.

Mouse rhodopsin genomic DNA sequences can be isolated from a mouse genomic DNA library, using methods known in the art (see Humphries et al., Nature Genet. 15:216-219 (1997) and Lem et al., Proc. Natl. Acad. Sci. USA 96:736-741 (1999)). Rhodopsin genomic DNA from other species can be obtained similarly. For example, a genomic library from a desired species can be screened with a probe from a rhodopsin cDNA from that species or, in view of the high degree of homology across species, a rhodopsin cDNA from another species, to isolate rhodopsin genomic DNA for use in a targeting construct. A restriction map of the genomic DNA can then be made, and suitable regions for insertion of the transgene determined.

The flanking homologous DNA sequences are of sufficient length for homologous recombination to occur between the targeting construct and an endogenous rhodopsin gene in a cell when the construct is introduced into the cell. Generally, the longer the homologous flanking sequence, the higher the efficiency of homologous recombination. An appropriate length of 5′ flanking sequence is at least about 1 kb, and is typically from about 1.5 kb to about 15 kb, such as from about 5 kb to about 10 kb. Likewise, an appropriate length of 3′ flanking sequence is at least about 1 kb, and is typically from about 1.5 kb to about 15 kb, such as from about 5 kb to about 10 kb.

The homologous sequences that flank the transgene are chosen so as direct the transgene to a desired position within the rhodopsin allele following homologous recombination. For example, if it is desired to drive expression of the transgene using native rhodopsin regulatory sequences, the 5′ homologous sequences can advantageously contain these sequences, such that the transgene will reside 3′ of the regulatory sequences in the recombined allele. The homologous regions that flank the transgene can also be chosen so as to make modifications, such as insertions, deletions and substitutions, in the recombined rhodopsin allele. For example, if it is desired to delete portions of the rhodopsin gene by homologous recombination (such as native 5′ regulatory elements, one or more exons, one or more introns), these regions are not included in the DNA sequences flanking the transgene. Deletions of portions of the endogenous rhodopsin gene are useful to ensure that a functional rhodopsin polypeptide is not expressed in the rod cells.

To provide for transcription and, ultimately, translation, of the transgene in rod cells, the construct is designed such that the transgene will be operably associated with rod-specific regulatory sequences following homologous recombination with a rhodopsin allele. As used herein, the term “operably associated” indicates that the rod-specific regulatory sequences and the transgene are positioned in such a manner so as to permit transcription of the transgene under the control of the rod-specific regulatory sequences.

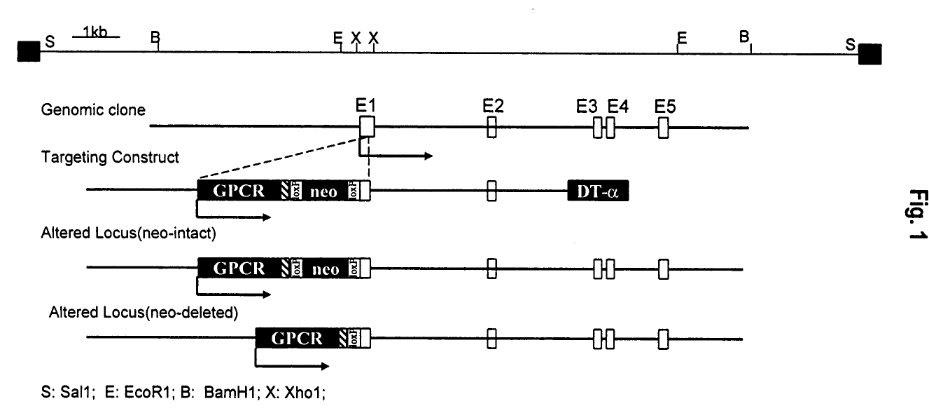

As used herein, the term “rod-specific regulatory sequences” refers to cis-acting DNA elements sufficient to direct transcription of the transgene in a rod cell. The term “rod-specific” means that the transgene is expressed at least in the rod cells, but does not require that the transgene be exclusively expressed in the rod cells. For use in a gene targeting construct, the rod-specific regulatory sequences are generally endogenous rhodopsin regulatory sequences included within the 5′ DNA sequence flanking the transgene (see FIG. 1). However, the rod-specific regulatory sequences can alternatively be rhodopsin regulatory sequences from other species, or regulatory sequences derived from other genes expressed in rod cells, such as arrestin, transducin ÿ, ÿ or ÿ subunits, phosphodiesterase ÿ, ÿ or ÿ subunits, or recoverin. Rod-specific regulatory sequences include promoter sequences that direct gene expression in the rod cells and, optionally, enhancer sequences that regulate the level of gene expression in the rod cells.

Regulatory sequences from rhodopsin genes are recognized by trans-acting factors in rod cells across species. For example, both bovine and human rhodopsin regulatory elements have been shown to direct expression of trangenes to mouse photoreceptor cells (Zack et al., Neuron 6:187-199 (1991); Nie et al., J. Biol. Chem. 271:2667-2675 (1996)). Rod-specific regulatory sequences can thus include regulatory elements of the rhodopsin gene from any vertebrate species (e.g. mouse, other rodent, bovine, Xenopus, human, pig, sheep, cat, dog, non-human primate, Zebrafish) and can include non-native DNA sequences.

Rhodopsin regulatory sequences, including promoter and enhancer elements, have been characterized in a number of species, including Xenopus (Mani et al., J. Biol. Chem. 28:36557-36565 (2001)), mouse (Lem et al., Neuron 6:201 -210 (1991)) and bovine (Nie et al., J. Biol. Chem. 271:2667-2675 (1996). These studies have indicated that fragments from −2174 to +70 bp; from −735 to +70 bp; from −222 to +70 bp; and from −176 to +70 bp, relative to the bovine rhodopsin mRNA start site, are able to direct photoreceptor-specific gene expression in transgenic mice (Nie et al., supra (1996)), indicating that the minimal cell-specific promoter lies within the region −176 to +70 bp of the bovine rhodopsin transcription start site. Likewise, 4.4 kb and 0.5 kb fragments from the mouse rhodopsin gene are able to direct photoreceptor-specific gene expression in transgenic mice (Lem et al., supra (1991)), indicating that the minimal cell-specific promoter lies within about 500 bp 5′ of the mouse rhodopsin transcription start site. Additionally, a highly conserved region of about 102 bp about 2 kb 5′ of the transcription start site of the bovine, human, mouse and rat rhodopsin genes has been identified as a transcription enhancer region (Nie et al., supra (1996)).

If desired, rod-specific regulatory elements can be modified from a native sequence to enhance tissue specificity or expression levels. For example, negative regulatory elements can be deleted so as to increase expression levels, without a change in rod cell specificity (Mani et al., supra (2001)). Additionally, multiple copies of enhancer elements can optionally be included, and sequences between the promoter and enhancer elements can optionally be deleted. Based on knowledge of rod-specific positive and negative regulatory elements, a skilled person can determine an appropriate sequence for directing expression of a transgene to rod cells.

A convenient assay for confirming that a particular regulatory sequence directs rod-specific gene expression takes advantage of the ease with which transgenic Xenopus can be made. A detectable reporter gene, such as green fluorescent protein or luciferase (or the desired transgene), can be operably linked to the candidate rod-specific regulatory sequence, and the construct introduced into Xenopus embryos by standard methods. Expression of the reporter gene (or the desired transgene) in the rod cells of the resulting tadpoles confirms that the regulatory sequence directs rod-specific gene expression (see Mani et al., supra (2001)).

The polypeptide expressed by the transgene also contains a rod outer segment (ROS) targeting signal to localize the polypeptide to the ROS membrane. Vertebrate rod cells consist of an outer segment that contains stacks of rhodopsin-containing disc membranes connected to the inner segment by a ciliary process. The inner segment contains the metabolic machinery of the cells, such as the mitochondria and Golgi. As used herein, the term “rod outer segment targeting signal” refers to a peptide sequence that confers localization of a heterologous polypeptide to the ROS membrane. An acceptable ROS targeting signal does not need to confer localization of the polypeptide exclusively to the ROS membrane. A small amount of expression of the polypeptide in other parts of the rod cell, including the inner segment, nucleus or synaptic body, will not be detrimental, as long as the polypeptide is abundantly expressed in the ROS membrane.

The necessary and sufficient features of vertebrate ROS targeting signals have been determined in transgenic Xenopus laevis by expressing chimeras between heterologous polypeptides and regions of X. laevis rhodopsin under the control of the X. laevis rhodopsin promoter. These studies have revealed that the C-terminal 8 amino acids of X. laevis rhodopsin (SSSQVSPA; SEQ ID NO:1)) are sufficient to confer outer segment membrane targeting on a heterologous polypeptide containing membrane association signals. Additionally, the 8 C-terminal amino acids of rhodopsin are sufficient for rhodopsin's vectorial transport to ROS through microtubule motor cytoplasmic dynein (see Tai, A. W., et al., Cell, 97:877-87 (1999)). When the rhodopsin C-terminus was attached to GFP, it was transported to ROS of rod cells of Xenopus (see Tam, B. M., et al., Cell Biol. 151:1369-80 (2000)) and zebrafish (see Perkins, B. D., et al., Vis Neurosci,. 19:257-64 (2002)). Also, rhodopsin-GFP fusions with the rhodopsin C-terminus attached was transported to ROS in Xenopus (see Moritz, O. L., et al., J. Biol Chem,. 276:28242-51(2001)) and mouse (see Wensel, T., et al., ARVO meeting (2001)). In addition to rhodopsin, other GPCRs are expressed in neuron rod cells. For example, 5-HT_{2A} receptor (see Pootanakit, K., et al., Vis Neurosci, 16:221-30 (1999)), cannabinoid CB1 receptor (see Straiker, A., et al., Proc Natl Acad Sci USA, 96:14565-70 (1999)), dopamine D2 receptor (see Nguyen-Legros, J., et al., J Neurochem, 67:2514-20 (1996)) and the metabotropic glutamate mGluR8 receptor. This neuronal prevalence corroborates that the biosynthesis machinery in rod cell is appropriate for GPCRs other than rhodopsin.

A peptide containing the C-terminal 25 amino acids of X. laevis rhodopsin (DEDGSSAATSKTEASSVSSSQVSPA; SEQ ID NO:2) also effectively confers outer segment membrane targeting on a heterologous polypeptide containing membrane association signals. These sequences were not sufficient, however, to confer ROS targeting on a cytoplasmic polypeptide (Tam et al., J. Cell Biol. 151:1369-1380 (2000)).

A longer sequence that contains the di-cysteine palmitoylation signal of rhodopsin, such as the C-terminal 44 amino acids of X. laevis rhodopsin (KQFRNCLITTLC*C*GKNPFGDEDGSSAATSKTEASSVSSSQVSPA; SEQ ID NO:3), is able to confer outer segment membrane targeting on a polypeptide that does not have its own membrane association sequences (Tam et al., supra (2000)). The two cysteine residues that are palmitoylated in the X. laevis C-terminal ROS sequence are indicated by asterisks.

ROS targeting signals can be recognized across species. For example, human rhodopsin can functionally rescue murine rod photoreceptors in rhodopsin knock-out mice (McNally et al., Hum. Mol. Genet. 8:1309-1312 (1999)). Therefore, a ROS targeting signal can be a naturally-occurring sequence from a rhodopsin from any vertebrate species (e.g. mouse, other rodent, bovine, Xenopus, human, pig, sheep, cat, dog, non-human primate, Zebrafish) or can be a non-naturally occurring sequence. The sequences of rhodopsins from a variety of species are known in the art (see, for example, GenBank gi: 129207 (human); gi:223659 (bovine); gi:129210 (mouse)). A ROS targeting signal can thus contain the native C-terminal sequence from a rhodopsin from any vertebrate species (e.g. mouse, other rodent, bovine, Xenopus, human, cat, dog, non-human primate; Zebrafish) or can be a non-naturally occurring sequence, such as a consensus sequence determined by aligning the ROS sequences from numerous species.

For example, a ROS targeting signal can include the eight (8) (ETSQVAPA; SEQ ID NO:4) or nine (9) (TETSQVAPA; SEQ ID NO:5) C-terminal residues shared by mouse, human and bovine rhodopsin, which are recognized by the rho I D4 monoclonal antibody (Molday et al., Biochemistry 22:653-660 (1983); MacKenzie et al., Biochemistry 23:6544-6549 (1994); Molday et al., Biochemistry 24:776-781 (1985)). Expression of a transgenic polypeptide containing the 1D4 epitope as the ROS targeting signal can advantageously be detected, and the polyepeptide isolated, by standard immunological assays using the rho 1D4 antibody. Another convenient ROS targeting sequence contains the 15 C-terminal residues from bovine rhodopsin (STTVSKTETSQVAPA; SEQ ID NO:6). Other suitable ROS targeting sequences correspond to the C-terminal amino acids (such as from about 8 to about 50 amino acids) of a vertebrate rhodopsin.

As described above with respect to rod-specific regulatory elements, a convenient assay for confirming the function of a candidate ROS targeting signal is to prepare transgenic Xenopus expressing the polypeptide/ROS fusion (and optionally further containing a detectable moiety) in their rod cells, and observing localization of the transgenic polypeptide to the rod outer segment membranes by microscopy (see, for example, Moritz et al., J. Biol. Chem. 276:28242-28251 (2001); and Tam et al., supra (2000)).

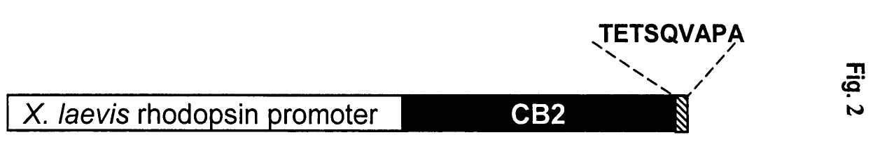

As an example, as shown in FIG. 2, the Xenopus laevis rhodopsin gene promoter can be inserted in front of a nucleotide sequence encoding the full-length human cannabinoid receptor 2 (CB2) fused to the 9 amino acid ROS targeting signal shown (SEQ ID NO:5). The construct can be transfected into Xenopus embryos and polypeptide expression in the ROS membrane of the tadpole confirmed by immunolocalization with a CB2 antibody.

A gene targeting construct can also contain one or more selectable markers. The construct generally contains at least one positive selection marker, the presence of which in the genome of a targeted cell indicates insertion of the construct into the genome, which can be random insertion or insertion by homologous recombination. Advantageously, the construct can also contain a negative selection marker, generally positioned at the 5′ or 3′ end of a linearized targeting construct, outside of the region of homology. The absence of the negative selection marker in the genome of the targeted cell, together with the presence of the positive selection marker, enriches for cells in which the construct has likely been inserted into the genome by homologous recombination rather than by random integration. Suitable positive and negative selection markers for gene targeting constructs can be selected by the skilled person, and methods for their use are well known in the art.

Positive selection markers include expressible genes that confer survival on a cell, such as genes that confer resistance to the drugs neomycin, hygromycin, puromycin or histidinol resistance. Alternatively, since ES cell lines are available that are deficient for hypoxanthin-phosphoribosyltransferase (HPRT), an expressible HPRT gene can serve as a positive selection marker and transfectants selected in HAT medium (Muller, Mech. Devel. 82:3-21 (1999)).

Negative selection markers include expressible genes that are directly or indirectly toxic to a cell. An exemplary negative selection marker is an expressible gene encoding the diphtheria toxin-A fragment (DTα). Another negative selection marker is the herpes simplex virus thymidine kinase (tk) gene that confers sensitivity to toxic nucleoside analogs such as gancyclovir or FIAU. An alternative negative selection marker is an expressible gene whose product can be recognized by an immunotoxic conjugate, such as the IL-2 receptor gene whose product is recognized by the recombinant immunotoxin anti-Tac (Fv)-PE40 (Muller, supra (1999); Kobayashi et al., Nucleic Acids Res. 24:3653-3655 (1996)).

In the exemplary gene targeting construct shown in FIG. 1, the homologous sequence 5′ of the transgene (which encodes a GPCR containing a C-terminal ROS targeting signal) contains about 1-5 kb of the mouse rhodopsin gene, including the native 5′ regulatory elements. The homologous sequence 3′ of the transgene contains about 1-5 kb, including part of exon 1 and optionally exon 2, of the mouse rhodopsin gene. The total length of the 5′ and 3′ homologous sequences are generally between 4 and 8 kb. The 5′ and 3′ homologous sequences are generally 1.5 kb or greater, and more usually 2 kb or greater, with the length depending, in part, on the availability of appropriate restriction sites. The construct also contains an expressible diphtheria toxin A gene (DTα) as a negative selection marker, and an expressible floxed neo gene 3′ of the trangene as a positive selection marker. Following homologous recombination between this construct and a mouse rhodopsin allele, the transgene and the floxed neo sequence will be inserted 3′ to the rhodopsin regulatory sequences so as to delete a portion of exon 1. The native mouse rhodopsin regulatory sequence thus directs expression of the transgene in rod cells, and the ROS targeting signal provides for localization of the encoded polypeptide in the ROS membrane. The insertion of the transgene at the rhodopsin allele functionally disrupts rhodopsin gene expression. Therefore, in an animal homozygous for the targeted allele, rhodopsin is expressed at low or undetectable levels, and the transgene is expressed in the ROS membrane.

The invention also provides a vector containing the gene targeting construct, and a host cell containing the gene targeting construct. A suitable vector can be a plasmid, cosmid, phage, BAC or other cloning vector into which large pieces of DNA can be inserted. The vector generally contains an origin of replication for amplifying the construct in a host cell. The vector can advantageously also contain a selection marker for selecting for host cells containing the vector. For amplifying the vector, the host cell will typically be a bacterial cell, but can alternatively be a yeast, insect, or mammalian cell. Methods of introducing a vector into a host cell are well known in the art (see, for example, Sambrook et al., supra (2001); Ausubel et al. supra (most recent supplement)).

Vectors suitable for use in gene targeting applications are available commercially (e.g. from Stratagene and Lexicon Genetics, Inc.). Dedicated gene targeting vectors conveniently include positive and negative selection marker s suitable for use in mammalian cells, together with appropriate cloning sites for inserting homologous gene sequences and transgenic sequences.

For certain applications, it is desirable to be able to remove the positive selection marker from the genome of a targeted cell or transgenic animal. Accordingly, a gene targeting construct can contain a positive selection marker operably positioned with respect to one or more sequences that facilitate its excision from the genome. Sequences suitable for facilitating excision of specific DNA sequences include recognition sites for site-specific recombinases. A variety of site-specific recombinases, including enzymes from bacteriophage, bacteria and yeast, and their recognition sites are known in the art (reviewed in Kilby et al., Trends in Genet. 9:413-421 (1993)). Those skilled in the art can choose appropriate sequences and corresponding enzymes for selective removal of the positive selection marker.

An exemplary system for specific DNA excision is the Cre/lox recombination system. The Cre/lox recombination system involves the use of the site-specific recombinase Cre (causes recombination) from phage PI that recognizes and binds to a 34-bp long, partly palindromic target sequence called loxP (locus of crossover x in P1). The loxP sequence is set forth as SEQ ID NO:7 (5′-ATAACTTCGTATAGCATACATTATACGAAGTTAT-3′). Cre recombinase has the ability to efficiently excise, by intramolecular recombination, any sequence placed between two loxP sites in the same relative orientation. A DNA sequence between two loxP sites in the same relative orientation is called a “floxed” sequence. As a result of Cre activity, one loxP site remains within the genome and one loxP site is is found on the excised circularized fragment (see FIG. 1; for reviews, see Muller, supra (1999); and Kilby et al., supra (1993)).

Methods are known in the art to excise a floxed DNA sequence, such as a floxed positive selection marker, from a targeted allele. One method is to transiently express Cre from an expression cassette in targeted embryonic stem (ES) cells, followed by screening ES clones to confirm deletion of the floxed sequence (see Xu et al., Genesis 30:1-6 (2001); Gu et al., Science 265:103-106 (1994)). An alternative method is to cross a transgenic mouse whose genome contains a floxed sequence with a transgenic mouse carrying the EIIa-Cre gene (Xu et al., supra (2001); Lakso et al., Proc. Natl. Acad Sci. USA 93:5860-5865 (1996)). A further alternative method is to inject a Cre-expression plasmid into the pronuclei of fertilized eggs from transgenic animals bearing the floxed sequence (Xu et al., supra (2001)). In the latter two methods, progeny mice in which the floxed sequence is deleted are identified by screening. Those skilled in the art can determine additional methods of removing a floxed sequence from a targeted allele.

In an invention transgenic construct, the transgene is flanked by 5′ upstream regulatory sequences of the rhodopsin gene from the animal species of interest. Conveniently, the animal species is a Xenopus. However, the invention can be practiced with other species amenable to transgenic procedures, such as mouse rat, guinea pig, bovine, Zebrafish, human, pig, sheep, goat, cat, dog and non-human primate. In one embodiment, the invention also provides a transgenic construct that contains rod cell specific cis-elements and a DNA sequence encoding a polypeptide comprising a rod outer segment (ROS) targeting signal. Following transgenic procedure, the transgene incorporates into the genome of the transgenic animal. An invention transgenic construct can advantageously be used, for example, to prepare animals that express the polypeptide encoded by the transgene in the rod outer segment membrane, and to prepare suitable cells (egg or sperm) for use in making such animals.

Vector constructs other than gene targeting constructs also are included as constructs and vectors useful in the methods of the invention. For example, non-target specific vectors can be used to generate transgenic animals for expression of a heterologous gene of interest. As described previously and further below, transgenic expression from random insertion into a host animal genome is well known in the art and can be used for transgenic production of essentially any desired polypeptide of interest that is amenable for expression in ROS employing the methods, constructs, vectors, cells and animals of the invention. An exemplary vector is shown in FIG. 2 for transgene expression in Xenopus. Non-targeting vectors offer advantages such as convenience and efficiency in the initial generation of transgenic animals as well as in the availability of wide variety of applicable vectors, species specific regulatory regions and animals amenable to efficient genomic insertion of a transgene.

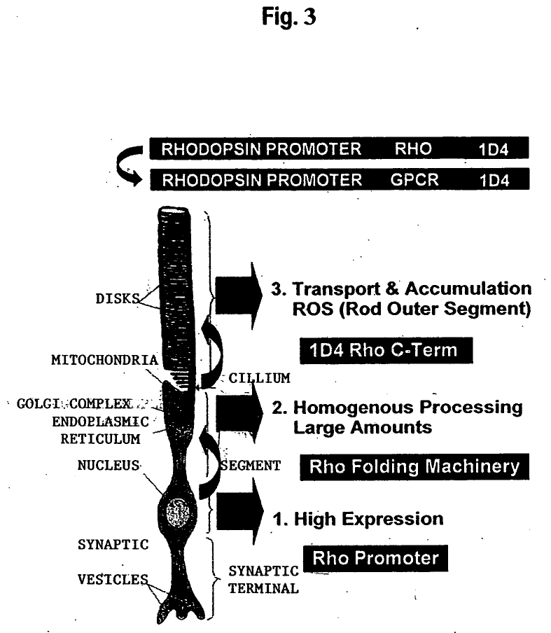

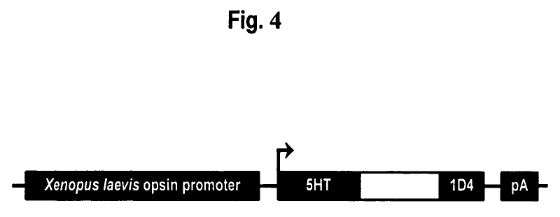

Transgenic vectors, such as the construct shown in FIG. 2, can efficiently introduce and effect expression of a heterologous gene into the genome of a host animal. To support expression in the ROS, all that is required is a rod specific regulatory region. Preferably, the rod specific regulatory region will also be species specific. Other exemplary vectors include those shown in FIGS. 3 and 4.

The invention also provides a cell whose genome contains a functional disruption of one or both endogenous rhodopsin alleles, and further contains a transgene encoding a polypeptide comprising a ROS targeting signal operably associated with a rod-specific regulatory sequence. Also provided is an animal whose genome contains a functional disruption of one or both endogenous rhodopsin alleles, and further contains a transgene encoding a polypeptide comprising a ROS targeting signal operably associated with a rod-specific regulatory sequence.

In certain embodiments, the cell is a mouse cell and the animal is a mouse. However, it is contemplated that the invention can be practiced with other species amenable to gene targeting or other transgenic procedures, such as rat, guinea pig, bovine, Xenopus, human, pig, sheep, goat, cat, dog, non-human primate or Zebrafish.

As used herein, the term “functional disruption” with respect to a rhodopsin allele means that the allele contains a mutation that prevents the normal function of the encoded polypeptide, such as a mutation that prevents expression of a normal rhodopsin polypeptide or that prevents expression of normal amounts of the rhodopsin polypeptide. The terms “functional disruption” and “knockout” are used herein synonymously. The mutation causing the functional disruption can be an insertion, deletion or point mutation.

In one embodiment, both rhodopsin gene alleles are functionally disrupted such that expression of the rhodopsin gene product is substantially reduced or substantially absent in cells of the animal. The term “substantially reduced” is intended to mean that less than 50% of the normal amount of rhodopsin is produced in rod cells of the animal, whereas the term “substantially absent” is intended to mean that essentially undetectable amounts of rhodopsin are produced in rod cells of the animal. Although animals with substantially reduced or substantially absent levels of rhodopsin are typically made by disrupting the coding region of the rhodopsin gene, an alternative approach is to disrupt the cis-regulatory elements of the gene such that transcription of the gene is down-regulated.

The skilled person will appreciate that there are various methods of making a cell or animal whose genome contains both a functional disruption of the rhodopsin gene and a particular transgene. For example, such a cell or animal can be obtained as a result of homologous recombination between a gene targeting construct containing the transgene and the endogenous rhodopsin gene, such that the transgene is inserted into a rhodopsin allele (called a “gene knock-in”). Alternatively, such a cell or animal can be obtained as a result of random insertion of the transgene into a rhodopsin gene knockout background, either directly or by cross-breeding a transgenic animal with a knockout animal.

An invention cell is intended to include a cell obtained prior to implantation into the animal (such as an embryonic stem cell, germ cell or embryo cell); a cell as it exists in the transgenic animal or its progeny; and a cell obtained or derived from the transgenic animal or progeny of said cell, such as an organ, tissue, isolated primary cell or established cell line.

An invention cell optionally expresses the transgenic polypeptide. For example, the cell can be a rod cell as it exists in a transgenic animal, or a rod cell isolated from a transgenic animal or progeny of said cell, such as an established rod cell line. Rod cells isolated from the transgenic animals of the invention generally express the transgenic polypeptide, due to the rod-specific regulatory elements directing transcription in the rod cells, as well as the ROS targeting signal which localize the polypeptide to the ROS membrane.

Suitable gene targeting constructs for use in a knock-in approach have been described above. Methods for preparing cells and animals using a gene targeting construct are well known in the art. Briefly, the targeting construct is introduced into an appropriate cell, such as an embryonic stem cell, by any of several techniques known in the art, including electroporation, calcium phosphate precipitation, DEAE-dextran transfection, microinjection, lipofection and the like. The cell is then cultured for a period of time and under conditions sufficient to allow for homologous recombination between the introduced targeting construct and an endogenous rhodopsin gene. Cells containing the inserted DNA are identified, such as by the positive or positive/negative selection methods described above. The selected cells can then be screened for homologous recombination at the endogenous rhodopsin gene locus by standard techniques, such as Southern hybridization or PCR using a probe or primer pair which distinguishes the endogenous allele from the recombinant allele.

If it is desired to create a cell homozygous for the rhodopsin gene disruption without a breeding step, drug escalation can be used (Mortensen et al., Mol. Cell. Biol. 12:2391-2395 (1992)) on the heterozygous cells. Alternatively, the first allele of a wild type cell can be disrupted by a first homologous recombination event that is selected with one marker (e.g. neomycin resistance) and then the second allele if the heterozygous cells can be disrupted by a second homologous recombination event that is selected with a different marker (e.g. hygromycin resistance).

To create a knock-in animal, an embryonic stem (ES) cell containing the recombinant allele is introduced into a blastocyst or aggregated with a morula, the blastocyst or morula is implanted into a pseudopregnant foster mother, and the embryo allowed to develop to term. The resultant animal is a chimera having cells descended from the embryonic stem cell. Chimeric animals in which the embryonic stem cell has contributed to the germ cells of the animal can be mated with wild type animals to produce animals heterozygous for the knock-in gene in all somatic and germ cells. The heterozygous animals can then be mated to create homozygous animals. Methods for obtaining, culturing and manipulating ES cells and other suitable cells for homologous recombination, and for preparing and identifying animals that are chimeric, heterozygous or homozygous for the recombinant allele, are known in the art and reviewed, for example, in Sedivy et al., Gene Targeting, W. H. Freeman and Co., New York (1992); Joyner (ed.) Gene Targeting: a Practical Approach. Oxford University Press, New York, 2^{nd} ed. (1998); and Ledermann, Exp. Physiol. 85:603-613 (2000)).

As an alternative to a knock-in strategy, the cells and animals of the invention can be made by introducing an appropriate transgenic construct into a genetic background in which the rhodopsin gene is functionally disrupted. Lines of mice with functional disruptions of the rhodopsin gene have been described in the art (see, for example, Humphries et al., Nature Genet. 15:216-219 (1997); Lem et al., Proc. Natl. Acad. Sci. USA 96:736-741 (1999)), and offspring of these mice can be obtained or additional lines of knockout animals prepared by similar methods.

A suitable construct for insertion of a transgene contains a DNA sequence encoding the transgenic polypeptide and ROS signal, operably linked to rod-specific regulatory sequences. Suitable polypeptides, ROS signals and rod-specific regulatory sequences have been described above. An exemplary rod-specific regulatory sequence for use in a transgenic construct is a 2.1 kb 5′ HindIII fragment from mouse rhodopsin (Geiger et al., Invest. Opthamol. Vis. Sci. 35:2667-2681 (1994)).

Methods for preparing transgenic animals are well known in the art. As an example of a typical method, the transgenic DNA construct is introduced into the male pronucleus of a fertilized egg (zygote), which is then implanted into a pseudopregnant female recipient animal. The embryo is grown to term, and offspring containing the transgene (heterozygous founder animals) are identified by Southern blotting or PCR. Different founder animals will have different sites of transgene integration, which can affect gene expression. Lines of animals with suitable expression of the transgenic polypeptide in rod cells can be identified and bred with wild-type animals to produce more animals with the same insertion (see Sedivy et al., supra (1992); Hogan et al., Manipulating the Mouse Embryo: A Laboratory Manual, 2nd ed., Cold Spring Harbor Laboratory (1994)). See also Capecchi et al., Science 244:1288 (1989); Zimmer et al., Nature 338:150 (1989); Shastry, Experentia, 51:1028-1039 (1995); Shastry, Mol. Cell. Biochem., 181:163-179 (1998); and U.S. Pat. No. 5,616,491, issued Apr. 1, 1997, U.S. Pat. No. 5,750,826, issued May 12, 1998, and U.S Pat. No. 5,981,830, issued Nov. 9, 1999).

As described herein, both targeted transgenic and non-targeted transgenic animals can be used for expression and production of heterologous polypeptides. For example, the Xenopus system described previously and shown in FIG. 2 can be used to produce a large number of transgenic tadpoles. Rod cells and ROS membranes from a population of tadpoles can be harvested and the transgene product isolated and purified. Essentially, any animal amenable to genomic insertion of a heterologously introduced gene and expression can be used for production of substantial quantities of isolated protein. Methods for transgenic expression for a variety of other species are well known in the art. Such other species include, for example, rat, guinea pig, bovine, Xenopus, Zebrafish, nematode, human, pig, sheep, goat, cat, dog and non-human primate. Advantages of non-targeted transgenic expression using include increased efficiency of production and avoidance of sequence specific constructs for implementation.

Both large and small populations of transgenic animals can be screened, as described previously and further below, for animals exhibiting sufficient transgenic expression for subsequent isolation of the transgenic product. Screens employing tags such as GFP or other marker as described herein can be employed for rapid and efficient identification of non-targeted transgenic animals. For example, Xenopus, nematode and other like animals can be gathered by the hundreds and ROS membrane fractions isolated for isolation of substantial quantities of transgenic polypeptides.

Constructs and vectors employed in non-targeted transgenic expression can consist of one or more regulatory elements sufficient to drive expression of the transgene and a ROS targeting signal for directing the transgene product to the ROS. Additionally, other markers, tags or modifications that impart beneficial or desirable characteristics on the transgenic expression system or on the transgenic product can additionally be employed. Detectable markers or tags such as GFP allow for efficient identification and isolation by visualization methods. Additionally, fluorescent tags such as GFP further allow for isolation of the transgenic product by methods such as fluorescent activated cell sorting (FACS). Particularly useful examples of constructs and vectors are described further below in the Examples and shown, for example, in FIGS. 2-4. These constructs can be employed with any transgene of interest for use in the methods of the invention and production of useful quantities of isolated or substantially pure polypeptides.

Additionally, given the teachings and guidance provided herein, those skilled in the art will understand that functional attributes useful for one transgenic system can be combined, substituted or modified with functional attributes of another transgenic system. For example, the construct components and configurations described herein or known in the art for targeted transgenic expression can similarly be employed for non-targeted transgenic expression. Similarly, construct components and configurations described herein or known in the art for non-targeted transgenic expression can similarly be employed for targeted transgenic expression. Accordingly, given the teachings and guidance provided herein, those skilled in the art will understand that numerous combinations of within and between transgenic expression systems can be mixed and matched to achieve desirable functional attributes or results. Accordingly, the constructs, vectors, cells and animals described herein are exemplary and intended to illustrate the compositions and methods of the invention.

Therefore, the invention provides constructs, vectors, cells and animals for both transgenic expression and targeted transgenic expression and production of heterologous polypeptides. The invention further provides methods and systems for production where a transgenic polypeptide is fused to a tag and expressed in a ROS of a host animal. Isolation of substantially purified polypeptide employing a cognate reagent to the tag allows efficient purification of the transgenic polypeptide. Such cognate reagents can be, for example, affinity binding reagents such as the 1D4 antibody described herein. Alternatively, such cognate reagents can be, for example, biophysical properties such as FAC sorting using fluorescent tags for the molecular identification. Various other methods and combinations of cognate reagents are well known in the art and can similarly be employed in the isolation methods of the invention.

Alternative methods known in the art can be used to introduce a transgene into animals to produce the founder lines of transgenic animals (see, for example, Hogan et al., supra, 1994; U.S. Pat. Nos. 5,602,299; 5,175,384; 6,066,778; and 6,037,521). Such methods include, for example, retrovirus mediated gene transfer into germ lines (Van der Putten et al., Proc. Natl. Acad Sci. USA 82:6148-6152 (1985)); electroporation of embryos (Lo, Mol Cell. Biol. 3:1803-1814 (1983)); and sperm-mediated gene transfer (Lavitrano et al., Cell 57:717-723 (1989)).

To make an animal with a transgene in a rhodopsin knockout background, generally a transgenic animal will be crossed with a knockout animal. Alternatively, the transgene can be introduced into a zygote containing the rhodopsin knockout allele, and the zygote grown to term as described above. By either method, offspring of the desired genotype are identified and additional animals produced by breeding.

The invention animals, whose genome contains a functional disruption of one or both endogenous rhodopsin alleles, and further contains a transgene encoding a polypeptide comprising a ROS targeting signal operably associated with a rod-specific regulatory sequence, can advantageously be used in a variety of applications. For example, large quantities of substantially purified transgenic polypeptide can be isolated from the outer segment membrane of rod cells of the eyes of the animals. For such purposes, animals in which the expression of endogenous rhodopsin is substantially absent due to functional disruption of both endogenous rhodopsin alleles are preferred, so that contamination of the ROS membrane with rhodopsin is minimized and purification is simplified. Additionally, intact rod cells and extracts thereof containing the transgenic polypeptide can be used in applications described herein.

In normal animals, about 90% of the protein content of the rod outer segment disc membranes is rhodopsin. In invention animals, due to rod cell-specific expression of the transgene and inclusion of the ROS targeting signal in the encoded polypeptide, it is expected that a substantial proportion (such as at least 10%, 25%, 50%, 75% or more) of the protein content of the rod outer segment disc membranes will instead be the transgenic polypeptide.

In normal animals, the typical yield of purified rhodopsin is about 0.1-1.0 nmol per mouse eye (Li et al., Proc. Natl. Acad. Sci. USA 92:3551-3555 (1995); Van Hooser et al., Proc. Natl. Acad. Sci. USA 97:8623-8628 (2000)). In invention animals, it is expected that a similar amount of transgenic polypeptide can be prepared from a similarly sized eye, with the actual amount depending on the animal species.

The skilled person can determine an appropriate method of substantially purifying a transgenic polypeptide from the rod cells of an invention animal. Generally, retinas are dissected from a suitable number of animals, and rod outer segments isolated as described by Papermaster et al., Methods Enzymol. 81:48-52 (1982) or Okada et al., Photobiol. 67:495-499 (1998). For example, retinas can be homogenized in a sucrose buffer, crude ROS sedimented by low-speed centrifugation, and substantially purified ROS isolated by density gradient centrifugation. For certain applications, it may be more convenient to use rod cell extracts, retinal extracts, or eye extracts as the starting source for substantially purifying the transgenic polypeptide.

The transgenic polypeptide can be solubilized from the ROS membrane using a suitable detergent. Solubilization conditions can advantageously be optimized so as to provide for single-step purification of the polypeptide. For example, alkyl(thio)glucosides with an appropriate hydrophilic-lipophilic balance (e.g. octylthioglucoside) in combination with a divalent cation provided for single-step purification of rhodopsin from ROS (Okada et al., supra (1998)). Alternatively, the solubilized polypeptide can be subjected to further purification using standard biochemical and immunological procedures, which can be chosen by the skilled person depending, for example, on the biological and immunological properties of the polypeptide and the degree of purity required for a particular application. Advantageously, a polypeptide containing a ROS targeting signal that contains the 1D4 epitope can be recognized by the 1D4 monoclonal antibody. Accordingly, the transgenic polypeptide can be isolated by standard immunoaffinity procedures known in the art.

The transgenic polypeptide can be obtained in sufficient concentration and purity so as to use to prepare a crystal suitable for structural analysis by X-ray crystallography. As described previously, as opposed to polypeptides expressed in tissue culture, polypeptides expressed in the ROS are relatively homogeneous with respect to post-translational modifications, which greatly facilitates crystallization. The conditions for generating high-quality crystals will depend on the polypeptide itself. However, exemplary conditions for preparing crystals from rhodopsin are described in Okada et al., J. Structural Biol. 130:73-80 (2000), and are expected to be relevant to many trangenic GPCRs as well as other transgenic polypeptides isolated from ROS membranes. Briefly, crystals can be prepared by hanging drop vapor diffusion from a solution containing at least about 5 mg/ml polypeptide in buffer containing about 30 mM MES or sodium acetate, 5-7 mM ÿ-mercaptoethanol, 65-90 Zn(OAc)₂, 0.55-0.75% HTPO, 0.45%-0.55% nonyl glucoside and 0.84-0.86 M ammonium sulfate. Alternative crystallization buffers and additives that can be used to improve crystallization are known in the art (see, for example, Rees et al., eds., Protein Engineering: A Practical Approach, Oxford University Press, Oxford (1992)).

A substantially purified transgenic polypeptide can also be used to prepare antibodies. Such antibodies can be advantageous in recognizing the polypeptide in its native form and with its native post-translational modifications. Optionally, for such purposes the transgenic polypeptide can be conjugated to a carrier protein and/or formulated together with an adjuvant to increase its immunogenicity, and used to immunize an appropriate animal. Methods of preparing polyclonal and monoclonal antibodies and antigen-binding fragments thereof (e.g. VL, VH and Fd; monovalent fragments, such as Fv, Fab, and Fab′; bivalent fragments such as F(ab′)_{2;} single chain Fv (scFv); and Fc fragments) and the like are described, for example, in Harlow and Lane, Antibodies: A Laboratory Manual, Cold Spring Harbor Laboratory, New York (1989); in Day, E. D., Advanced Immunochemistry, Second Ed., Wiley-Liss, Inc., New York, NY (1990); and in Borrebaeck (Ed.), Antibody Engineering, Second Ed., Oxford University Press, New York (1995).

A further application for substantially purified transgenic polypeptides is in the preparation of pharmaceuticals. For example, if the transgenic polypeptide is an antibody, it can be conjugated to a toxin and administered to an individual to specifically target cells expressing the corresponding antigen, such as tumor cells. A a further example, if transgenic polypeptide is a receptor agonist or antagonist, it can be administered to an individual to modulate receptor signaling associated with a pathological condition. Pharmaceutical applications for various polypeptides are known in the art or can be determined. The substantially purified polypeptide can be formulated together with a pharmaceutically acceptable excipient. The amount of polypeptide and the precise formulation will depend on the nature and biological activity of the polypeptide, as well as the intended route of administration. Suitable methods and excipients for formulating pharmaceuticals are desribed, for example, in Remington's Pharmaceutical Sciences (Maack Publishing Co., Easton, Pa., most recent edition).

The transgenic polypeptide can also be used in drug screening applications. For example, rod cells, ROS membrane extracts, or substantially purified polypeptides can be contacted with a candidate compound, and the ability to the compound to bind the polypeptide determined. A compound that binds the polypeptide is a candidate ligand, agonist, antagonist or reverse agonist of the polypeptide. The functional effect of the compound can subsequently be determined by functional assays appropriate to the particular polypeptide. Suitable candidate compounds for use in screening assays include chemical or biological molecules such as simple or complex organic molecules, metal-containing compounds, carbohydrates, peptides, proteins, peptidomimetics, glycoproteins, lipoproteins, nucleic acids, antibodies, and the like, and libraries of such compounds can readily be prepared or are commercially available. Various binding assays, including high-throughput binding assays are known in the art and can be used in screening assys, including scintillation proximity assays (SPA), UV or chemical cross-linking, competition binding assays, biomolecular interaction analysis (BIA), surface plasmon resonance (SPR), mass spectrometry (MS), nuclear magnetic resonance (NMR), and fluorescence polarization assays (FPA). The skilled person can determine appropriate compounds and assays for a particular screening application.

Intact cells from a transgenic animal that express the transgenic polypeptide, including rod cells within the animal retina and rod cells isolated from the animal, can also be used in drug screening assays, including binding assays similar to those described above and function-based screening assays. Appropriate function-based screening assays will depend on the normal function of the polypeptide. For example, if the transgenic polypeptide is a receptor, signaling through the receptor in response to the compound can be determined. Exemplary signaling assays depend on the nature of the receptor, but can include, for example, determining altered production or turnover of a second messenger, NTP hydrolysis, influx or efflux of ions or amino acids, altered membrane voltage, increased or decreased protein phosphorylation, altered activity of an enzyme, altered protein-protein interactions, relocalization of a protein within the cell, or induction of gene expression. For certain functional assays in which the relevant effector molecules or reporter genes are not normally present in rod cells, the animal genome can be further modified by knock-in or transgenic methods so as to express these components. The effect of naturally occurring and man-made mutations on transgenic polypeptide activity can likewise be determined by function-based assays.

Additional applications for the transgenic animals, cells and substantially purified transgenic polypeptides of the invention can be determined by those skilled in the art.

It is understood that modifications which do not substantially affect the activity of the various embodiments of this invention are also included within the definition of the invention provided herein. Accordingly, the following examples are intended to illustrate but not limit the present invention.

### EXAMPLE I

This example shows the construction of a gene targeting construct to replace mouse rhodopsin in the retina with a G-protein coupled receptor.

A genomic fragment containing all five exons of mouse rhodopsin and its regulatory elements is obtained by the method described in Humphries et al., Nature Genetics 15:216-219 (1997). Briefly, a rhodopsin cDNA probe is used to isolate a clone containing a 129Sv-derived mouse genomic fragment from a ÿ phage library. A restriction map of this fragment showing relevant restriction sites is shown in FIG. 1 (top). An 11 kb BamH1 fragment derived from the initial genomic fragment is subcloned into a pKO Scrambler V907 vector (Lexicon Genetics, Inc.) to generate the genomic clone shown in FIG. 1.

A transgenic cassette containing a G-protein coupled receptor cDNA tagged at its C-terminus with a 1D4 tag and a neomycin resistance gene flanked by two loxP sites is first constructed by standard molecular biology methods. Briefly, by PCR the termination codon of the GPCR cDNA is replaced by a sequence encoding, in-frame, the 9 amino acid 1D4 epitope tag (TETSQVAPA; SEQ ID NO:5) followed by a stop codon.

The tagged GPCR is ligated to the ploxP-neo-loxP (“floxed neo”) cassette prepared as described in Yang et al., Proc. Natl. Acad. Sci. USA 95:3667-3672 (1998). The GPCR/floxed neo cassette is then ligated into the rhodopsin genomic clone between the rhodopsin promoter and exon 1, deleting part of the exon 1 coding sequence, such that expression of the GPCR is driven by the rhodopsin promoter.

A diphtheria toxin A chain cassette (Labarca et al., Proc. Natl. Acad. Sci. USA 98:2786-2791 (2001)) is also ligated at the 3′ end of the construct to provide the targeting construct shown in FIG. 1. The construct thus contains both an excisable positive selection marker (neo) and a negative selection marker (DTÿ) for use in selecting for homologous recombination in ES cells.

Targeting constructs suitable for replacing the mouse rhodopsin gene with other transgenes are made by similar methods.

### EXAMPLE II

This example shows the construction of a gene targeting construct to replace mouse rhodopsin in the retina with the human cannabinoid receptor 2.

The human cannabinoid receptor 2 (CB2) cDNA (Genbank Accession No. X74328) is cloned from a human spleen cDNA library. The 9 amino acid 1D4 tag is added to the C-terminus of CB2 by PCR using the sense primer 5′-GCC GCC ACC ATG GAG GAA TGC TGG GTG AC (SEQ ID NO:8) and the anti-sense primer 5′-TTA GGC TGG AGC CAC CTG GCT GGT CTC CGT CTT GGA AGC GGT GGC AGA G (SEQ ID NO:9). The junction is sequenced to confirm that the CB2/l D4 fusion is in-frame. A neomycin resistance cassette (neo), with a phosphoglycerate kinase promoter and polyadenylation signal and flanked by loxP sites, is inserted downstream of the CB2/1D4 fusion. The targeting constructed is created by replacing the DNA segment between the Xho1 sites of the rhodopsin gene with the CB2-neo cassette, deleting 15 bp upstream of the translation start site and the first 111 codons of the rhodopsin gene. The diphtheria toxin A chain (DTα) genie with the RNA polymerase II promoter is inserted at the 3′end of the targeting construct to provide negative selection.

### EXAMPLE III

This example shows the introduction of a gene targeting construct into embryonic stem (ES) cells and the production of transgenic mice.

The gene targeting construct described in Example I or II is electroporated into 129 Sv ES cells, and the ES cells are cultured in the presence of the neomycin analog G418. Correctly targeted ES clones, which have an altered rhodopsin locus as shown in FIGS. 1 or 2, are resistant to G418. Incorrectly targeted clones are killed by expression of the DTÿ gene. DNA from G418 resistant clones is screened to confirm homologous recombination, by PCR analysis and Southern blotting.

The ES cells are transiently transfected with a Cre recombinase expression vector, such as a cytomegalovirus-Cre plasmid, and Cre-mediated excision of the neo^{r} gene at the flanking lox sites confirmed by sequence analysis of the PCR-amplified gene segment.

Correctly targeted ES cell clones with the neo^{r} gene excised are microinjected into C57BL/6 blastocysts, which are then implanted into pseudopregnant female mice. Chimeric male offspring are identified by their mixed coat color and bred to females, and offspring heterozygous for the targeted allele identified by PCR analysis and Southern blotting. Heterozygotes are then cross-bred to produce homozygous mice.

The homozygous mice produce the transgenic polypeptide instead of rhodopsin in the outer segment membrane of rod cells.

### EXAMPLE IV

This example describes the production of transgenic Xenopus laevis tadpoles expressing several recombinant polypeptides in rod outer segments of retina rod cells.

As described previously, the retina rod cells can be converted to a bioreactor for expression of a polypeptide of interest, where every second 80,000 new rhodopsin polypeptides are synthesized. This example describes the conversion of rod cells into a bioreactor to produce several different GPCR polypeptides. In order to convert the native rhodopsin expression system into a bioreactor for the GPCR of interest, a useful DNA construct can be a rhodopsin promoter driving the expression of the DNA encoding the GPCR fused to the 1D4 ROS targeting signal. This construct and the roles of each component are shown in FIG. 3.

Rod cells perform three steps to become a useful source for expression or expression and purification of heterologous polypeptides such as the 5-HTR GPCRs described in this example. These steps are shown schematically in FIG. 3. First, the endogenous rhodopsin 5′ regulatory sequence drives expression of the 5-HTR or other heterologous encoding gene selectively in these rod cells. Second, the endogenous biochemical machinery of rod cells, which can process rhodopsin polypeptide at a rate of 50,000 rhodopsin proteins per second, processes the substituted 5-HT GPCR or other heterologous nascent polypeptide with reasonable or comparable rates of efficacy. Maintaining reasonable processing efficiency results in the production of large quantities of properly folded and relatively homogenous receptor, including glycosylation pattern. Finally, the 1D4 ROS-targeting signal fused at the C-terminus of the 5-HT GPCR, corresponding to the C-terminus of rhodopsin, drives transport of properly folded 5-HT receptor to the ROS where it accumulates in disk membrane sacks much like rhodopsin.

Transgenic Xenopus laevis tadpoles were generated using the construct shown in FIG. 3, adding Green Fluorescence Protein to facilitate visualization (GPCR-GFP-1D4), by injection into Xenopus oocytes. As described further below, the results obtained show successful expression of all 12 5HTRs in the ROS of transgenic tadpoles as judged by GFP expression. Furthermore, the 5HT_{1A}R was characterized from these tadpole samples, and shown to properly fold by ligand binding and GTPγS activation, and homogenously glycosylated.

Transgenic tadpoles expressing each of the 12 human 5HT GPCRs (5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆, or 5TH₇ receptors) in the photoreceptors of retina rod cells were produced by injecting transfected sperm nuclei into unfertilized eggs. Xenopus laevis offer some advantages over transgenesis in other animals because the transgene is integrated into the male genome prior to fertilization, resulting in embryos that are not chimeric, which a voids subsequent breeding steps. Another advantage is that the embryos are easily manipulated and accessible immediately after fertilization, enabling quick verification of GPCR expression in rod cells and their accumulation in ROS in only 1-2 weeks. FIG. 4 shows the final construct for each 5HTR, which is composed of five fragments consisting of: (1) a Xenopus rhodopsin promoter fragment, which is used to direct expression of the fused protein (see Mani, S. S., et al., J. Biol Chem, 276:36557-65 (2001)); (2) a 5HT receptor encoding segment; (3) GFP (green fluorescent protein) is fused for visualization of transgenic tadpoles; (4) a 15-residue-long C-terminus region from mouse rhodopsin (RHO15) that contains the ROS-targeting signal termed 1D4, and (5) a polyadenylation site.

The above construct for transgenic expression and methods for producing and assessing the resultant transgenic tadpoles are described below. Briefly, the coding sequences the 12 5HT receptors cDNAs were derived from available sources. For example, the sequences for 5HT_{1A} (Genbank accession number M83181), 5HT_{1B} (M81590), and 5HT_{1E} (M91647) receptors were amplified from a pool of human genomic DNA (Novagen) by PCR using primers for the coding sequence of receptors.). cDNAs of 5HT_{1F} (AF498981), 5HT_{2A} (X57830), 5_{HT}_{2B} (X77307), 5HT_{2C} (M81778) 5HT₆ (L41147) and 5HT_{7A} (U68487) receptor were cloned from a pool of human brain mRNA by RT-PCR using primers complementary to portions of these Genbank sequences. Receptors 5HT_{4A} (Q13639), 5HT_{5A} (P47898) and 5HT_{1D} (M89955) cDNA clones were purchased from Aptus Bioscience and Invitrogen, respectively. The 5HT 1, 2, 5 and 6 subfamilies do not have any reported splice variants and the generated cDNAs were verified by sequencing against the Genebank Reports for each of the receptors. The 5HT4 and 5HT7 receptors have multiple isoforms. The most predominantly expressed isoform for each of these receptors was chosen for transgenic expression. The PCR products were generated using taqplus DNA polymerase (Stratagene), cloned into pCRII vector (Invitrogen), and sequenced to ensure that the sequences are identical to the ones deposited in Genbank.

The Xenopus laevis expression constructs were constructed using the expression plasmids pXOP-5HTR-GFP-1D4. Briefly, the pXOP-C1-GFP vector was cut by Agel/AccIII to remove the GFP sequence, and re-ligated to generate pXOP-C1 minus plasmid. A DNA fragment encoding the last 15 amino acids of mouse rhodopsin (1D4) and the GFP sequence were inserted into pXOP-C1 minus to produce pXOP-N1-GFP-1D4. The cDNAs encoding 5HT receptors were amplified from the vectors pCRII-5HTRs, respectively, using sequence-specific primers, and the Kozak translation initiation sequence was added to the initial codons of these fragments. The amplified products were inserted into the srfl site of pXOP-N1-GFP-1D4 to generate pXOP-5HTR-GFP-1D4 expressing plasmids. The resulting plasmids were sequenced to confirm the correct coding frame.

Transgenic Xenopus laevis embryos were generated by intra-cytoplasmic sperm injection (ICSI). The transgene fragment was released from the vector by restriction digestion, and purified from agarose gel using the Bio 101 Geneclean Spin kit (Qbiogene). Sperm nuclei were permeabilized using digitonin. The protocol for ICSI was performed as described by Sparrow et al. (see Sparrow, D. B., et al. Nucleic Acids Res 28:E12 (2000)), with minor modifications, using snap frozen sperm nuclei (see Zhang, L., et al., Development, 130:4177-86 (2003)). Specifically, about 400,000 sperm nuclei (in 4 μl) were incubated with 250-500 ng transgene DNA (in 2.5 μl water) at room temperature for 15 minutes. The reaction mixture was diluted with 22.5 μl sperm dilution buffer (SDB, 250 mM sucrose, 75 mM KCl, 0.5 mM spermidine trihydrochloride, 0.2 mM spermine tetrahydrochloride), then 2.5 μl of the diluted mixture was transferred to 230 μl SDB for injection. Eggs were injected in 0.4 MMR (Marc's Modified Ringer's, 100 mM NaCl, 2 mM KCl, 2 mM CaCl₂, 1 mM·MgCl₂, 75 mM HEPES) containing 6% (w/v) Ficoll. Properly gastrulating embryos were raised in 0.1 XMMR until approximately stage and then transferred to dechlorinated tap water. Tadpoles were anesthetized in 0.01% 3-aminobenzoic acid ethyl ester (Sigma) and monitored for GFP (green fluorescence protein) expression using a Leica MZFL III fluorescent stereoscope. Developmental stages of embryos were determined according to Nieuwkoop and Faber, Normal Table of Xenopus laevis (1967).

Microscopy of transgenic Xenopus laevis eyes was performed by fixing transgenic tadpoles in freshly-prepared fixative (2% paraformaldehyde, 2% glutaraldehyde in 0.1 M phosphate buffer, pH 7.3) for 3 hours at 4° C., washed in 5%, 10%, 15%, and 20% SPB (sucrose phosphate buffer) series, then equilibrated in 20%SPB/OCT (2:1) at 4° C. for overnight, and finally embedded in 20%SPB/OCT (2:1). Embedded transgenic tadpoles were cryosectioned through the eye. The sections were counterstained with Hoechst 33342 to reveal retina structure. Green fluorescent protein expression was visualized under a Nikon fluorescent microscope.

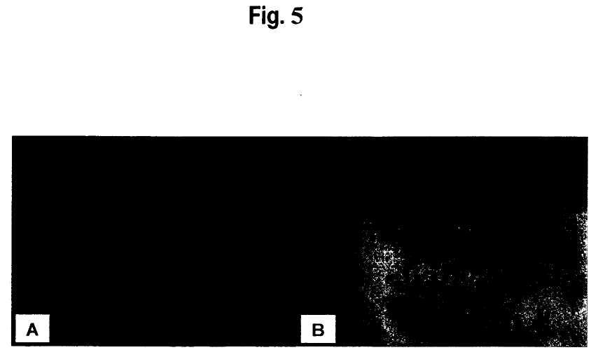

The results obtained demonstrate successful transgenic expression of all 12 human 5HTRs in the ROS of transgenic tadpoles. Generally, the linearized transgene construct was incubated with sperm nuclei and then injected them into several hundred Xenopus laevis oocytes. The percentage of dividing embryos varied from 20-45%, depending more on the quality of Xenopus eggs and specific conditions than on the particular 5HTR construct injected. The presence of green fluorescence was studied among tadpoles that survived 7 days or longer (development stage 48 or later). As shown in FIG. 5, green fluorescence was observed in the eyes of Xenopus tadpoles for all 12 5HTRs, indicating successful expression of all 12 5HTR-GFP-1D4 fusion proteins. Panel A shows a fluorescence field image of the eyes of transgenic tadpole as stained above. Panel B shows a lateral view of the transgenic tadpole eye. The picture is a combination of a bright field image and a fluorescence field image

Substantial levels of fluorescence intensity and transgenic efficiencies were observed among these 12 5HTRs. Transgenic production efficiencies are shown in FIG. 6 where transgenic efficiency (a) corresponds to the percent GFP expressing tadpoles among surviving tadpoles after stage 42. The relative intensity (b) corresponds to the average intensity relative to the strongest expresser 5HT5A. One of the highest intensities, indicating the highest receptor expression, was observed for 5HT_{1A}, which also has a high transgenic efficiency. Therefore, this receptor was selected for further characterization as described below. The intensity of green fluorescence varied among different tadpoles, but in most cases it was stronger than in transgenic Xenopus tadpoles expressing only soluble GFP fused to the C-terminus of rhodopsin (see Tam, B. M., et al., Cell Biol. 151:1369-80 (2000)).

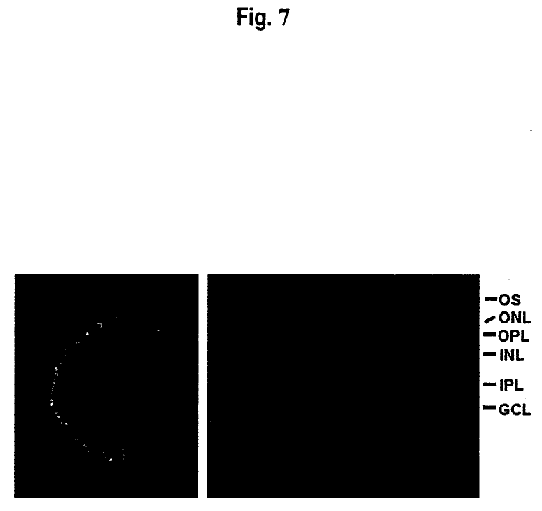

Cryosection of retina from transgenic tadpoles was performed to confirm the extent of restricted expression in retinal rod cells, and within these rod cells the extent of ROS targeting for these GPCR-GFP-1D4 constructs. Transgenic tadpoles were collected after stage 48 when they had fully differentiated functional retina. The retinas of transgenic tadpoles were cryosectioned to reveal the location of 5HTR-GFP expression. The sections were counterstained with hoechst dye that labels cell nuclei to show the nuclear layer organization in the retina. The results are shown in FIG. 7. Localization of GFP is shown by green areas, relative to the blue areas that identify the cell nuclei. Panel A snows the cross section of entire retina of the tadpole eye for the 5HT_{1A} GPCR at 20× magnification, while panel B shows a 100× amplified section to discern the targeting of the GFP signal relative to the nuclei of the rod cells (OS: outer segment; ONL: outer nuclear layer; OPL: outer plexiform layer; INL: inner nuclear layer; IPL: Inner plexifrom layer, GCL: ganglion cell layer).

Expression 5HT_{1A}R-GFP-1D4 was consistent across the back of the eye that defines the retina as shown in FIG. 7 (green), and specific to the rod cells within the entire eye. The transgenic tadpole retina displays a mosaic expression pattern of fusion protein. While the expression level of fusion protein varies between individual tadpoles, it also showed some variability from one rod cell to another within the same retina. This finding is consistent with the similar mosaic expression pattern of 1D4-tagged rhodopsin-GFP fusion protein in Xenopus retina using the same promoter reported previously (see_Moritz, O. L., et al., J. Biol Chem,. 276:28242-51 (2001)). Cell-to-cell expression variability may be the result of position-effect variegation (see Moritz, O. L., et al., J. Biol Chem,. 276:28242-51 (2001)). As can be seen in the amplified section on FIG. 7, the 5HT_{1A}R-GFP-1D4 polypeptides are specifically expressed in the ROS of the rod cells, without any discernable expression in the rest of the rod neurons. This result indicates that the opsin promoter selectively drives expression of these 5HT_{1A}R-GFP-1D4 constructs on retinal rod cells, and that the 1D4 (or RHO 15) targeting signal from mouse rhodopsin effectively and selectively targets receptor transport into the rod outer segments (ROS).

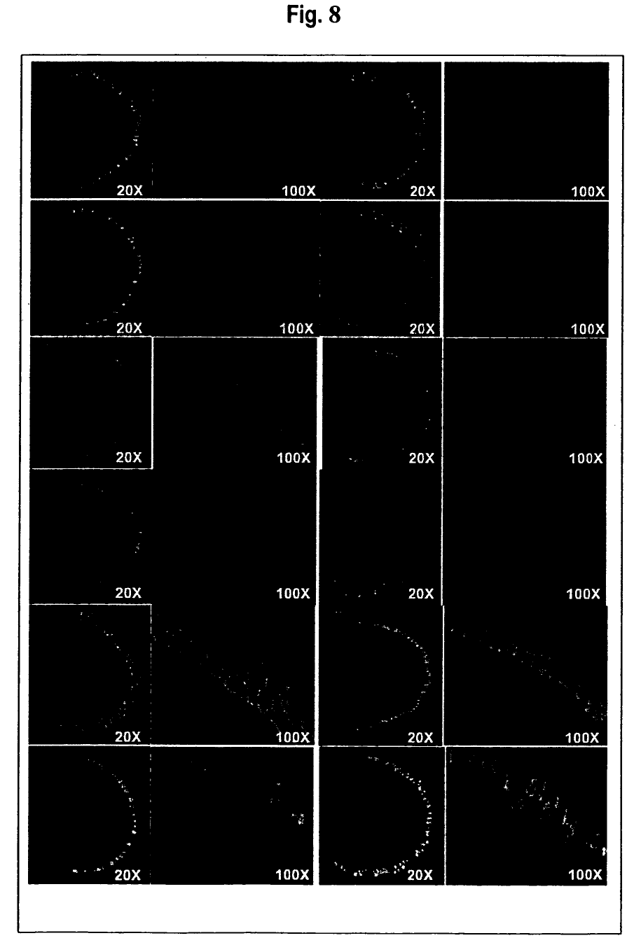

Similar results to those described above for 5HT_{1A}R-GFP-1D4 have been obtained with all other 11 5HTRs. The results for all 12 5HTRs are shown in FIG. 8 where the pairs of panels A-L correspond to a 20× magnification and a 100× amplification of the cryosections, respectively, as described above for FIG. 7. Panel pairs shown from left to right are the results for 5HT_{1A (panel A)}, 5HT_{1B (panel B)}, 5HT_{1D (panel C)}, 5HT_{1E (panel D)}, 5HT_{1F (panel E)}, 5HT_{2A (panel F)}, 5HT_{2B (panel G)}, 5HT_{2C (panel H)}, 5HT_{4A (panel I)}, 5HT_{5A (panel J)}, 5HT_{6 panel K)}, 5HT_{7A (panel L)}. For all 12 5HTRs GFP signal was specifically detected in the eye of the tadpole, and within the eye in the rod neuron. Some of 5HTRs fusion proteins, such as 5HT_{1A}, 5HT_{1D}, 5HT_{2B}, 5HT_{5A}, 5HT₆ and 5HT₇ receptors, were expressed exclusively and at high levels within the ROS.

The above results show that transgenic constructs convert retina of Xenopus laevis tadpoles into a bioreactor for all 5HTRs tested. GFP fusion protein accumulation was not detected in ER and Golgi apparatus, indicating that all the fusion protein is properly folded and transported to the ROS disc membranes, and without contamination from breakdown products containing GFP. This result is in contrast to receptor overexpresion results obtained in mammalian cells, which in general lead to intracellular accumulation of unfolded receptor protein in the ER and Golgi. Furthermore, this result further corroborates that the rhodopsin folding machinery is present at high levels in these rod cells to fold the large amounts of rhodopsin being produced because misfolded protein would not be efficiently transported to the ROS.

Fusion construct expression and immunoaffinity isolation also was performed to confirm that the 15-residue-long C-terminus of mouse rhodopsin fused to the human 5-HT receptors is accessible to the 1D4 antibody and available for immunoaffinity purification of the heterologously expressed polypeptide. This immunoaffinity purification is a specific illustration of the methods described previously and is applicable to all heterologously expressed transgenes of the invention. General application and exemplification of this two-step purification procedure is described further below in Example V.

In this specific study, cell lines were generated using HEK293 cells transiently expressing each of the 12 human 5-HT receptors fused to the 15-residue-long C-terminus of mouse rhodopsin. The T7 tag, corresponding to the N-terminus from the major capsid protein from the T7 bacteriophage, was added between the receptor and the 1D4 tag. Because the 1D4 antibody also binds to mouse rhodopsin, the T7 tag could also can be useful for detection and/or purification of the receptors from animals co-expressing endogenous rhodopsin.

Briefly, solubilization and deglycosylation of the receptors from transfected cells was performed using about 25-50 million of HEK 293T cells transiently expressing one h5HTR-T7-1D4. Transfection was performed using procedures well known to those skilled in the art. Following harvesting, cells were washed with Tris buffer saline (TBS) and frozen until use. For the receptor purification, the cells were thawed on ice and subjected to hypotonic shock with 5 mM Tris pH 7.5 buffer (containing a protease inhibitor cocktail, DNase, and 1 mM MgCl₂). The membranes were washed with 10× TBS, and resuspended in 1 mL of 2× TBS. Then 0.75 mL of 50 mM dodecyl maltoside (DM) was added, the tube was rocked for 15 min at 4° C., and spinned down at 21,000 g for 30 min. A 50-μL aliquote of the supernatant was saved, and the rest was incubated with 5 μL of PNGase F (SIGMA) for 12 h at 16° C. Completion of the deglycosylation was assessed by western blotting.

Purification was performed by adding 100 μL of immobilized-1D4 antibody in sepharose gel to the sample and rocked for 30 min at 4° C. The gel and the sample were loaded onto a small column, and the gel was washed with 5 mL of washing buffer (30 mM sodium acetate pH 5.5, 5 mM DM, 1 M NaCl). The elution was carried out by adding a competing nonapeptide to the washing buffer and collecting 5×100-μL fractions. The second or third elution fraction, which generally contained higher concentration of receptor, was compared with the crude aliquot by electrophoresis and silver staining.

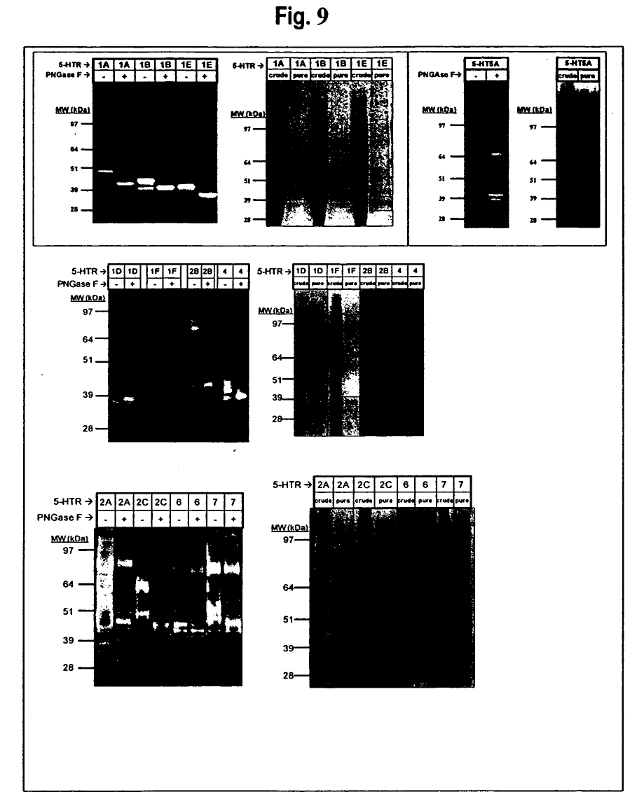

The immunoblottings comparison from the above procedures are shown in FIG. 9. For each receptor the left lane shows the crude extract of membrane proteins and the right lane represents the same extract treated with PNGase F. Antibody 1D4 was used as a primary antibody. The silver stained panels show purified receptors with immobilized 1D4 antibody (right lanes) compared with crude extracts. In each case a box shows the main band(s).

The results indicated that all the receptors were glycosylated in a more or less heterogeneous manner. After incubation with PNGase F, most of the receptors showed a single monomer band (accompanied by a fainter dimer band, as usual for other GPCRs). In some cases, an extra band close to the deglycosylated monomer and other minor bands (probably due to post-translational modifications or incomplete deglycosylation) were present. This level of purification was sufficient for further study since the purified receptors could be easily identified in a main band by electrophoresis.

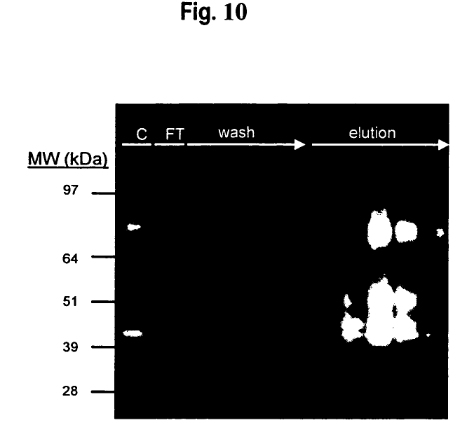

In some cases, due to low expression and/or poor cell growth, the amount of purified receptor was very low. In these cases, the best elution fraction was identified by Western blot and it was concentrated by microfiltration before loading on the gel fur silver staining (see 5-HT₆R as an example in FIG. 10; C: deglycosylated crude; FT: flow-through). Receptors were purified to significant levels in a single immunochromatographic step. However, in all cases, a band (or double band) could be identified as the receptor by comparing the electrophoretic mobility in the immunoblotting. The calculated molecular weights were in all cases 85-90% of the expected from the sequence.

These results show that the expressed receptors (h5HTR-T7-1D4) have their 1D4 tag accessible to the immobilized 1D4 antibody when solubilized in detergent micelles, and they can be affinity purified as demonstrated by competition with a peptide representing the epitope.

One of the human 5-HT receptors expressed in Xenopus rod cells described above was further characterized by quantification of receptor expression, intracellular localization, SDS-PAGE and immunoblotting, and functional binding assays with a specific ligand. Isolation and purification of transgenically expressed 5HT_{1A} was performed as described above. Briefly, tadpole eyes were collected for Western blot and ligand-binding assays by anesthetizing transgenic tadpoles with relatively strong GFP expression and washing in PBS. The eyes were dissected out, collected, and stored at −80° C. Solubilization of the 5HT_{1A} receptor was performed from the eyes of 305 tadpoles expressing the fusion protein h5HT_{1A}R-GFP-1D4, washed with PBS and frozen until use. For the receptor purification, the eyes were thawed and sample was treated as described above for the transfected HEK293 cells, except that no deglycosylation step was included. Immunoblotting with different antibodies was carried out to confirm identity of the receptor. Purification of the receptor with immobilized 1D4 antibody in sepharose gel was similarly performed as described above for the transfected HEK 293 cells.

Radioligand binding assay were performed using transgenic tadpoles (stage 48 or older) or CHO cells expressing 5HT_{1A} receptors homogenized in binding buffer (50 mM Tris-HCl, pH 7.4, 2 mM MgCl₂, 1 mM EDTA and proteinase inhibitor cocktail). The binding assay was performed in 96-well plate with [methyl-³H]-MPPE as a radioligand. For saturation binding analyses, the membranes (20 μg/well) were incubated with 0.05% saponin in a final volume of 50 μl at room temperature for 10 min, and then incubated with 50 μl of [methyl-³H]-MPPE (0-50 nM) for another 90 min. Nonspecific binding was determined in the presence of 10 μM serotonin. Assays were terminated by rapid filtration through GF/C filter plate with 4 times washes with washing buffer (20 mM Trics-HCl, pH 7.4). The radioactivity was measured by a TopCount (PerkinElmer). Assays were performed in duplicate.

A receptor binding assay was performed to determine retention of functional receptor using [³⁵S]GTPγS labeled receptor ligand. Transgenic tadpoles (stage 48 or older) or CHO cells expressing 5HT_{1A} receptors were homogenized in binding buffer (20 mM HEPES, pH 7.4, 10 mM MgCl₂, 1 mM EDTA, 100 mM NaCl). The binding assay was performed in 96-well Scintiplate (PerkinElmer). To reduce [³⁵S]-GTPγS binding to rhodopsin-activated transducin in retina membrane, the membrane from tadpoles was preincubated with 10 mM hydroxylamine in binding buffer at room temperature for 60 min. The membranes (25 μg/well) were incubated with indicated ligands for 30 min in a total volume of 150 μl binding buffer containing 10 μM GDP, 0.5% BSA, 1 mM DTT), then 50 μl of 800 μM [³⁵S]-GTPγS was added to each well to make a final concentration of 200 pM. The binding assays were carried out at room temperature for 60 min under gentle shaking. Final concentration of hydroxylamine in binding assay should be no more than 1 mM, Nonspecific binding was determined in the presence of 10 μM unlabelled GTPγS. Assays were terminated by centrifugation at 4000 g for 15 min and removal of supernatant. The radioactivity was measured by a TopCounter (PerkinElmer). Assays were performed in triplicate.

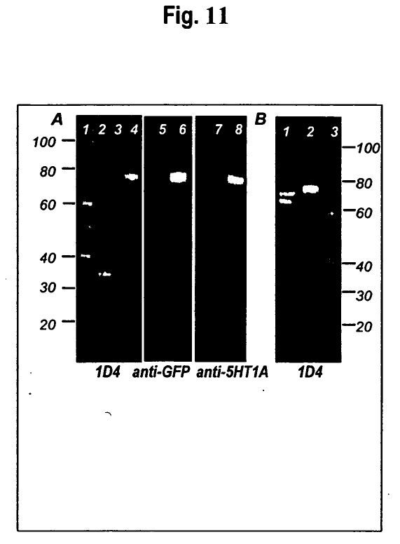

Following the above procedures, the identity of 5HT_{1A}R-GFP-1D4 fusion protein was confirmed by 3 parallel western blots incubated with different antibodies. The results are presented in FIG. 11 and show a band present in the crude extract that binds to anti-GFP, anti-5HT_{1A}, and 1D4 antibodies. Panel A shows membrane extracts of negative controls (lanes 3,5,7) or transgenic (lanes 4,6,8) Xenopus tadpole eyes that were probed with 1D4 (lane 1-4), anti-GFP (lane 5-6) or anti-5HT_{1A} antibody (lane 7-8) (lane 1: BenchMagic Marker; Lane 2: bovine rhodopsin. Panel B shows 5HT1AR-GFP-1D4 in transgenic Xenopus eyes that was detected by western blot before (lane 2) and after (lane 1) it was incubated with PNGase F (lane 3: BenchMagic Marker).

In the blot incubated with 1D4 antibody no signal was observed due to rhodopsin, in spite of their abundance in the crude sample. This result shows that the Xenopus rhodopsin C-terminal sequence is different to the mammalian one and it is not recognized by 1D4 antibody. The mouse sequence is SATASKTETSQVAPA while the Xenopus sequence is ATSKTEASSVSSSQVSPA, having an insertion of a SVSSS sequence in sixth position from the C-terminus. Regardless of this immunogenic difference, the 1D4 tag from mouse that the Xenopus receptor construct carries is recognized as a ROS targeting signal by the transport machinery of frog rod cells and transported efficiently to the ROS. This result further corroborates that the band at ˜70 kDa corresponds to the fusion protein h5HT_{1A}R-GFP-ID4.

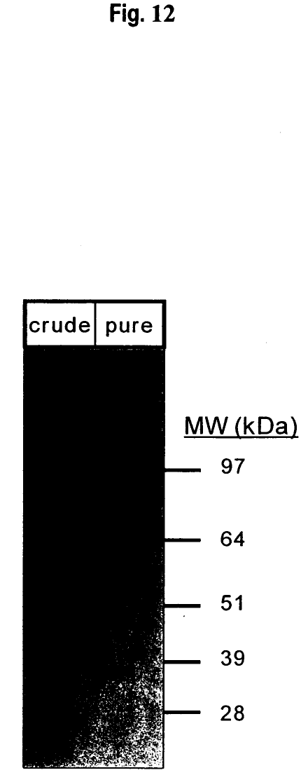

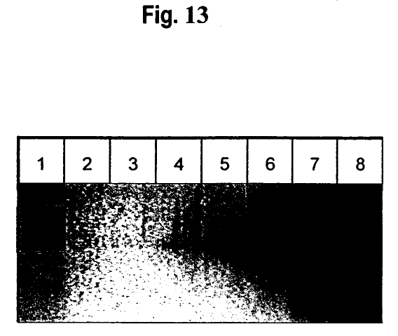

The electrophoresis was developed by silver staining to compare the purified receptor compared with the crude extract. FIG. 12 shows a silver stained gel of total membrane protein of transgenic tadpole eyes (left lane) and purified 5HT1A-GFP-1D4 (right lane). Only a minor contaminant was observed after a single chromatographic step (most probably frog rhodopsin, which is abundant in the extract). The expression level of the fusion protein is quantified in FIG. 13 by comparison with bovine serum albumin. Shown is an aliquot of purified receptor corresponding to about 7.5 tadpoles loaded onto the gel (box in lane 1) and compared to increasing amounts of BSA corresponding to 0.21, 1.1, 5.5, 27, 136, 680, and 3400 ng (lanes 2 to 8). Comparison of the transgenic polypeptide expression with the BSA standard curve yielded an estimate that each transgenic tadpole expresses about 1-5 ng of fusion protein.

Binding assays were performed to confirm that the 5HT_{1A} receptor was expressed as a functional 5HT_{1A}R-GPF-1D4 fusion protein in rod cell ROS of Xenopus tadpoles. Binding affinity was determined by measuring the binding affinity of [methyl-³H]-MPPF, a radiolabeled selective ligand to the 5HT_{1A} receptor, to the expressed fusion protein. Because the 5HT_{1A} receptor expressed in ROS disk membranes is oriented with the extracellular side facing the interior of these disks and shielded from the solution, the detergent saponin was used to open up pores in these disk membranes and allow access to the ligands. Saponin at a concentration of 0.05% or more could effectively increase specific binding of [methyl-³H]-MPPE to 5HT_{1A}R transgenic tadpole membranes, but not to control tadpole membranes. This result indicated that saponin could effectively open up pores in the disk membranes to allow access of 5HT_{1A} receptors to the ligands. Based on these observations, 0.05% saponin was included in all ligand binding assays.

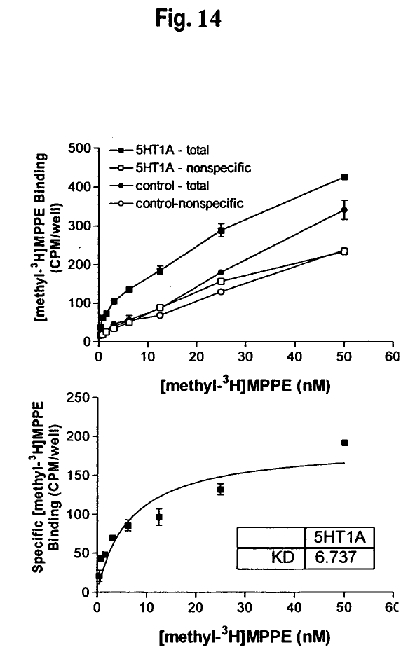

FIG. 14 (top) shows the specific binding of transgenic 5HT_{1A} tadpole membranes to [methyl-³H]-MPPE in a dose-dependent manner, whereas control tadpole membranes did not show significant specific binding up to 25 nM of [methyl-³H]-MPPE. This result indicates that the heterologous receptor is expressed and folded because it can recognize its selective ligand. Saturation binding assays were also performed to measure the binding affinity and compare it to 5HT_{1A} receptor expressed in standard mammalian CHO cells. The results of the saturation binding experiments of transgenic tadpoles expressing the 5HT_{1A}R-GFP-1D4 are shown in FIG. 14 (bottom). The binding affinities of [methyl-³H]-MPPF on these tadpole samples was K_{D}=6.7 0.7 nM, while the equivalent measurements performed on membrane samples from CHO cells expressing the 5HT_{1A} receptor was K_{D}=0.35±0.1 nM. There was an about 17-fold difference in the dissociation constants K_{D} between transgenic tadpoles and CHO cells. The low nanomolar affinity and extent of specific binding further corroborates that the 5HT_{1A} receptor is properly folded in transgenic tadpoles. However, the 17-fold difference indicates that factors such as the different lipid composition of their respective membranes may have a minor effect on binding or conformation. For example, the effect on binding affinity may be due to different ligand kₒₙ or k_{off} kinetic parameters, or different partition coefficients to the different lipids present in these membranes.

To show that 5HT_{1A} receptors expressed in Xenopus rod cells can functionally interact with endogenous G proteins in retina rod cells, the binding of [³⁵S]GTP□S to Xenopus laevis tadpole membranes was determined. To reduce rhodopsin-mediated [³⁵S]GTP□S binding to transducin in ROS membrane, rhodopsin was inactivated by treatment of tadpole membrane with 10 mM hydroxylamine at room temperature. This treatment led to about a 50% reduction of basal [³⁵S]GTP□S binding to tadpole membranes without significant effect on the [³⁵S]GTP∇S binding to CHO membranes, corroborating specific inactivation of rhodopsin-transducin interaction in ROS membranes.

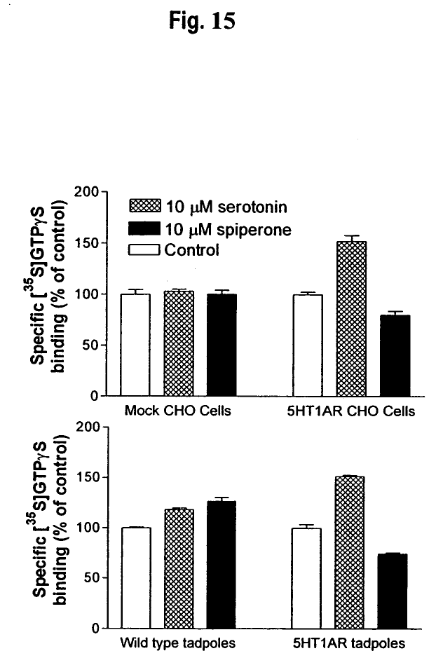

The results of these G protein coupling studies are shown in FIG. 15, where the agonist serotonin specifically stimulated, while the inverse agonist spiperone specifically inhibited, [³⁵S]GTPγS binding to 5HT1ARs in both CHO cells and transgenic tadpoles. Briefly, agonist serotonin stimulated [³⁵S]GTP□S binding to tadpole membranes by about 50%. This result was similar to that observed from CHO membranes transiently expressing 5HT_{1A}. The inverse agonist spiperone, specific for 5HT_{1A}, could significantly block [³⁵S]GTP□S binding to both CHO membranes and tadpole membranes, indicating that 5HT_{1A} displayed the constitutive Gᵢ-coupling activity in both CHO cells and Xenopus rod cells. These results indicate that 5HT_{1A} expressing in Xenopus rod cells could properly couple to G proteins similar to receptors expressing in mammalian cells.

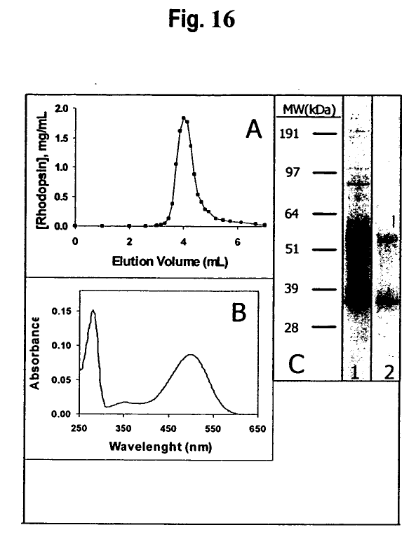

The iochemical folding machinery of rod cells is particularly useful to produce purified receptor protein for crystallography or other purposes because of the observed chemical homogeneity of the folded receptors transported to the ROS with respect to posttranslational modifications, especially glycosylation. For example, the heterogeneous patterns for 5-HT receptors expressed in HEK293 cells can be converted to a more homogenous pattern by deglycosylation (FIG. 9), indicating that there was a high degree of heterogeneous glycosylation in the receptors purified from those cells. In contrast rhodopsin extracted from native rod cells shows a tight electrophoretic band (see FIG. 16C below), suggesting it is homogenously glycosylated. In fact, rhodopsin is glycosylated at two residues and substantially all of the purified rhodopsin from rod cells is biglycosylated without contamination from significant amounts of mono- or non-glycosylated protein. Moreover, the chemical composition of the actual glycan added to each residue also is substantially homogenous. This lack of contamination from unglycosylated protein is due to the intracellular purification step that occurs in rod neurons, where only properly folded and homogenously glycosylated rhodopsin is transported to the ROS, where it forms 90% of all the membrane protein content. This step results in an intracellular purification of rhodopsin in neurons for enrichment of homogenous material. Separation of rod outer segment (ROS) structures from the rest of the rod cells in the above studies shows the intracellular purification step because significant levels of unglycosylated rhodopsin is not observed. The separation between finally folded receptor from unprocessed receptor being folded is another advantage of the homogeneity of purified receptor sample from these rod cells relative to standard cell lines.

### EXAMPLE V

This example illustrates a general method of the invention for transgenic expression and purification of heterologous polypeptides, and specifically for GPCR transgenic expression and purification.

A universal two-step method for the purification to homogeneity of GPCRs, such as the 5-HT receptors, expressed in the rod outer segment of mouse, Xenopus or other animal retina is described below. The method is described with reference to rhodopsin as a model GPCR and takes advantage of two attributes of gene targeted transgenic expression such as the mouse expression system described previously. First, rhodopsin comprises more than 90% of the protein content of rod outer segment. A reliable method based on sucrose gradient centrifugation is available for isolating rod outer segment from retina extracts (see Papermaster, D. S., Methods Enzymol, 81:48-52 (1982)). This method can be employed with mouse or other retina using Optiprep as the density gradient medium (see Tsang, S. H., et al., Science, 282:117-21 (1998)). This rod outer segment enrichment protocol can be used as a first step for GPCR purification. Second, as described previously, GPCRs can be C-terminal tagged with rhodopsin's ROS-targeting sequence, which allows transport of the protein to the ROS. The monoclonal antibody 1D4 monoclonal antibody shows a high affinity for this sequence and has been extensively used for the purification of rhodopsin and other 1D4-tagged GPCRs. Although a ROS-targeting sequence is exemplified in this method, it is understood that essentially any immunoaffinity tag can be fused to the constructs of the invention to facilitate subsequent affinity purification. Such tags can be fused in addition to an ROS targeting signal at a location in the fusion construct that does not destroy function of the transgenic fusion product or the function of a ROS targeting signal. Alternatively, a ROS targeting signal—cognate affinity binding molecule pair can substitute for the 1D4 signal and cognate 1D4 specific antibody. Such affinity tags, cognate affinity binding partners and functional substitutions or combinations thereof are well known in the art.

Preparation of an immunoaffinity column was performed by attaching the monoclonal antibody specific to the 1D4 tag, which corresponds to the nine C-terminal residues of rhodopsin. Monoclonal antibody was produced by 1D4 hybridoma cells using methods well know in the art. The antibody was purified to homogeneity in two chromatographic steps. First, 4 L of hybridoma supernatant were dialyzed against 10 mM Tris-Cl, pH 8, loaded in a column containing 60 g of DEAE cellulose, and eluted with a gradient of 0-0.5 M NaCl in 10 mM Tris-Cl, pH 8. The fractions were analyzed by SDS polyacrylamide gel electrophoresis (PAGE), and those fractions containing antibody were pooled and diluted 1:1 with protein-A binding buffer (ImmunoPure® (A) IgG Binding Buffer from Pierce). This antibody solution was loaded in a 15-mL rProtein-A Sepharose column (Pharmacia) and eluted with 0.1 M glycine pH 2.8. The fractions were immediately neutralized with 10% of 1 M Tris-Cl pH 8. Finally, the antibody was dialyzed against phosphate buffered saline (PBS) buffer and coupled to a cyanogen bromide-activated-agarose matrix (AminoLink Plus Gel from Pierce) at a density of 5 mg antibody per mL of the gel.

For the rhodopsin purification, all procedures were performed under dim red light. The first step consisted of ROS enrichment from dark-adapted bovine retina extracts by sucrose gradient centrifugation (see Papermaster, D. S., Methods Enzymol, 81:48-52 (1982)). Then, the ROS preparation was solubilized in pH 7.4 buffer containing 20 mM dodecyl-maltoside and loaded in a 20-cm long 1D4 affinity column. Elution was achieved by addition of 0.1 mM of a competing peptide representing the nine C-terminal residues of rhodopsin (TETSQVAPA). Purity of the most concentrated fractions was assessed by absorbance and SDS-PAGE.

The results of this two-step purification procedure are shown in FIG. 16. Panel A shows the chromatographic profile of rhodopsin eluted with a competing peptide from a 20-cm long column of immobilized 1D4 antibody. Panel B shows an absorbance spectrum of rhodopsin in ground state where 50 μL of the most concentrated fraction in panel A was diluted in 1 mL of hydroxylamine-containing buffer. Panel C shows a SDS-polyacrylamide gel electrophoresis of purified rhodopsin (lane 1: ROS extract; lane 2: most concentrated fraction in panel A).

Rhodopsin purified by 1D4 immunoaffinity chromatography showed a ratio A₂₈₀/A_{500=1.6}-1.7 as shown in FIG. 16B, indicating that rhodopsin in this sample was pure and functional. Moreover, only two bands, corresponding to the monomer and dimer, were observed when the sample was run in SDS-PAGE and stained with Coomassie (FIG. 16C). Rhodopsin purified on a 20-cm long column by immunoaffinity chromatography reaches about 2 mg/mL in the most concentrated fractions (FIG. 16A). After concentrating 3-4 times in a centrifugal concentrator, the rhodopsin concentration was sufficient for crystallization or other uses.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims. All journal article, reference and patent citations provided above, in parentheses or otherwise, whether previously stated or not, are incorporated herein by reference in their entirety.

1. A transgene construct, comprising a transgene encoding a membrane polypeptide comprising a rod outer segment (ROS) targeting signal, and an operably associated species specific rod-specific regulatory sequence, wherein said polypeptide is not rhodopsin.

2. The construct of claim 1, wherein said membrane polypeptide is a G protein-coupled receptor (GPCR).

3. The construct of claim 2, wherein said GPCR comprises a receptor selected from 5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆, 5HT_{7A}, EDG1, EDG2, EDG3, EDG4, EDG5, EDG6, EDG7, EDG8, CB2, FMLP and MC4.

4. The construct of claim 1, wherein said membrane polypeptide is a fusion protein.

5. The construct of claim 1, wherein said ROS targeting signal comprises SEQ ID NO:4.

6. The construct of claim 1, wherein said rod-specific regulatory sequence comprises a rhodopsin promoter.

7. The construct of claim 4, further comprising a tag sequence.

8. The construct of claim 7, wherein said tag sequence comprises GFP.

9. The construct of claim 1, wherein said species specific rod-specific regulatory sequence comprises a Xenopus laevis opsin promoter.

10. A vector comprising the construct of claim 1.

11. A cell comprising the construct of claim 1.

12. A Xenopus cell whose genome comprises a transgene encoding a membrane polypeptide comprising a ROS targeting signal operably associated with a Xenopus rod-specific regulatory sequence, wherein said polypeptide is not a rhodopsin.

13. The Xenopus cell of claim 12, further comprising a functional disruption of one endogenous rhodopsin gene alleles.

14. The Xenopus cell of claim 12, wherein said membrane polypeptide is a GPCR.

15. The Xenopus cell of claim 14, wherein said GPCR comprises receptor selected from 5HT_{1A}, 5HT_{1B}, 5HT_{1D}, 5HT_{1E}, 5HT_{1F}, 5HT_{2A}, 5HT_{2B}, 5HT_{2C}, 5HT_{4A}, 5HT_{5A}, 5HT₆ and 5HT_{7A}, EDG1, EDG2, EDG3, EDG4, EDG5, EDG6, EDG7, EDG8, CB2, FMLP and MC4.

16. The Xenopus cell of claim 12, wherein said membrane polypeptide is a fusion protein.

17. The Xenopus cell of claim 12, wherein said ROS targeting signal comprises SEQ ID NO:4.

18. The Xenopus cell of claim 16, wherein said membrane polypeptide further comprises a tag sequence.

19. The Xenopus cell of claim 18, wherein said tag sequence comprises GFP.

20. The Xenopus cell of claim 13, wherein said genome comprises a functional disruption of two or more endogenous rhodopsin gene alleles.

21. The Xenopus cell of claim 12, further comprising a sperm cell or an egg.

22. The Xenopus cell of claim 12, further comprising a rod cell.

23. An extract of the Xenopus cell of claim 12, comprising an outer segment membrane of said cell.

24. A Xenopus tadpole or adult whose genome comprises a transgene encoding a membrane polypeptide comprising a ROS targeting signal operably associated with a Xenopus rod-specific regulatory sequence, wherein said polypeptide is not a rhodopsin.

25. The Xenopus tadpole or adult of claim 24, further comprising a functional disruption of one endogenous rhodopsin gene alleles.

26. The Xenopus tadpole or adult of claim 24, wherein said polypeptide is a GPCR.

27. The Xenopus tadpole or adult of claim 26, wherein said GPCR comprises receptor selected from 5HT_{1A}, 5HT_{1B}, 5HT_{1D}, 5HT_{1E}, 5HT_{1F}, 5HT_{2A}, 5HT_{2B}5HT_{2C}, 5HT_{4A}, 5HT_{5A}, 5HT₆ and 5HT7A, EDG1, EDG2, EDG3, EDG4, EDG5, EDG6, EDG7, EDG8, CB2 FMLP and MC4.

28. The Xenopus tadpole or adult of claim 24, wherein said polypeptide is a fusion protein.

29. The Xenopus tadpole or adult of claim 24, wherein said ROS targeting signal comprises SEQ ID NO:4.

30. The Xenopus tadpole or adult of claim 28, wherein said polypeptide further comprises a tag sequence.

31. The Xenopus tadpole or adult of claim 30, wherein said tag sequence comprises GFP.

32. The Xenopus tadpole or adult of claim 25, wherein said genome comprises a functional disruption of two or more endogenous rhodopsin gene alleles.

33. A rod cell, or outer membrane extract thereof, isolated from the Xenopus tadpole or adult of claim 24.
